(19) United States
(12) Patent Application Publication
(10) Pub. No.: US 2006/0004076 A1
(43) Pub. Date: Jan. 5, 2006

(54) Co-administration of dehydroepiandrosterone (DHEA) congener with pharmaceutically active agents for treating inflammation

(75) Inventors: Dinesh Patel, Salt Lake City, UT (US); David Vollmer, West Jordan, UT (US); Claire Daugherty, Salt Lake City, UT (US); John Gebhard, Salt Lake City, UT (US)

Correspondence Address: THORPE NORTH & WESTERN, LLP., 8180 SOUTH 700 EAST, SUITE 200, SANDY, UT, 84070, US

(73) Assignee: Inflabloc Pharmaceuticals, Inc.

(21) Appl. No.: 11/145,024

(22) Filed: Jun. 3, 2005

Related U.S. Application Data
(60) Provisional application No. 60/584,350, filed on Jun. 30, 2004.

(51) Int. Cl. A61K 31/415; A61K 31/202

(52) U.S. Cl. 514/406; 514/560; 514/547

(57) ABSTRACT

The present invention is related to therapeutic uses of dehydroepiandrosterone (DHEA) congeners. More specifically, the present invention relates to the co-administration of a dehydroepiandrosterone (DHEA) congener in combination with at least one other pharmaceutically active agent to reduce inflammation.

The present non-provisional application claims the benefit of U.S. Provisional Application No. 60/584,350 filed Jun. 30, 2004, which is incorporated herein by reference.

### FIELD OF THE INVENTION

The present invention is drawn to methods of reducing inflammation. More particularly, the present invention relates to the co-administration of a dehydroepiandrosterone (DHEA) congener in combination with at least one other pharmaceutically active agent to reduce inflammation.

### BACKGROUND OF THE INVENTION

Inflammation within a human subject is a common physiological response by the immune system to an injury or irritation, where the irritation can be by infectious, allergic, and/or chemical irritants. Some of the clinically observable symptoms of inflammation include increased redness, temperature, swelling, and pain, as well as the loss of function within the inflamed area. These symptoms can be a direct result of infiltration of body fluids and leucocytes (white blood cells) into the inflamed area. This physiological response can be beneficial for the subject because of the ability of the body fluids to dilute any present toxins or substances, to facilitate the entry of antibodies, nutrients, oxygen, and immunological cells to the site, and to aid in drainage from the site. Additionally, leucocytes can aid in destroying any foreign substance within the inflamed area.

While inflammation can primarily be a favorable defense mechanism, it can also have unfavorable consequences when it is an inappropriate immunological response incited by a non-harmful substance. Additionally, diseases such as human inflammatory disorders, infectious disorders, and autoimmune disorders can also result in unfavorable inflammation.

An abbreviated listing of some inflammatory diseases includes arthritis, bronchitis, allergic rhinitis, atopic dermatitis, chronic cholecystitis. Additionally, inflammation arising from a disease or an inappropriate immunological response can have other physiological effects that may not be desirable including fever, malaise, nausea, enlarged lymph nodes, increased erythrocyte sedimentation, and leucocytosis. As such, most subjects do not want to suffer from any diseases or inappropriate immunological responses that result in inflammation, and desire treatment and/or prevention of such inflammation and associated maladies. Thus, research and development continues to seek pharmaceutical products for reducing inflammation.

### SUMMARY OF THE INVENTION

It has been recognized that it would be advantageous to provide compositions and methods for reducing inflammation in a subject, and inhibiting other associated maladies. As such, the present invention provides for methods of reducing inflammation in a subject. One of these methods can include co-administering a therapeutically effective amount of a DHEA congener and a second anti-inflammatory agent to the subject. In another aspect, a method of reducing inflammation in a subject can include co-administering a therapeutically effective amount of a DHEA congener and an anti-TNF-α agent to the subject. Compositions suitable for carrying out these methods are also provided.

Additional features and advantages of the invention will be apparent from the detailed description, which illustrates, by way of example, features of the invention.

### BRIEF DESCRIPTION OF THE DRAWINGS

FIG. 1 is a graphical representation depicting the effect of DHEA and sulfasalazine alone, as well as in combination, on in vivo TNF-α in a TNBS IBD animal model;

FIG. 2 is a graphical representation depicting the myeloperoxidase levels from the same animal model; and

FIG. 3 is a graphical representation depicting the effect of DHEA and ibuprofen alone, as well as in combination, on in vivo paw swelling in a collagen induced arthritis animal model.

### DETAILED DESCRIPTION OF THE INVENTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and “the” include plural referents unless the context clearly dictates otherwise. Thus, for example, reference to “a drug” includes reference to one or more of such drugs.

As used herein, the terms “formulation” and “composition” may be used interchangeably and refer to a combination of a pharmaceutically active agents, such as a DHEA congener formulated with one or more additional anti-inflammatory agent(s). The terms “drug,” “active agent,” “bioactive agent,” “pharmaceutically active agent,” and “pharmaceutical,” can also be used interchangeably to refer to an agent or substance that has measurable specified or selected physiologic activity when administered to a subject in an effective amount. These terms of art are well known in the pharmaceutical and medicinal arts.

As used herein, “administration,” and “administering” refer to the manner in which a drug, formulation, or composition is introduced into the body of a subject. Administration can be accomplished by various art-known routes such as oral, parenteral, transdermal, inhalation, implantation, etc. Thus, an oral administration can be achieved by swallowing, chewing, or sucking an oral dosage form comprising active agent(s). Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. These and additional methods of administration are well known in the art.

The term “co-administering,” co-administration,” or “co-administer” refers to the administration of a DHEA congener with another anti-inflammatory agent. Both the DHEA congener and the second anti-inflammatory agent can be administered simultaneously, or at different times, as long as these active agents work in concert to produce a physiological effect. Additionally, co-administration does not require the DHEA congener and the second anti-inflammatory agent to be administered by the same route. As such, each can be administered independently or as a common dosage form.

The terms “effective amount,” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. In some instances, a “therapeutically effective amount” of a drug can achieve a therapeutic effect that is measurable by the subject receiving the drug. The determination of an effective amount is well within the ordinary skill in the art of pharmaceutical, medicinal, and health sciences. See, for example, Meiner and Tonascia, “Clinical Trials: Design, Conduct, and Analysis,” Monographs in Epidemiology and Biostatistics, Vol. 8 (1986), which is incorporated herein by reference.

As used herein, the terms “inhibit” or “inhibiting” refers to the process of holding back, suppressing or restraining so as to block, prevent, limit, or decrease a rate of action or function. The use of the term is not to be misconstrued to be only of absolute prevention, but can be a referent of from any incremental step of limiting or reducing a function to the full and absolute prevention of the function. In one example, when the term “inhibit” is utilized in combination with a substance, such as an immune mediator responsive to TNF-α, inhibition of the production of the substance can include the reduction of the production and/or secretion of the substance. Alternatively, TNF-α receptors can be inhibited from receiving TNF-α.

As used herein, “reduce” or “reducing” refers to the process of decreasing, diminishing, or lessening, as in extent, amount, or degree of that which is reduced. The use of the term with respect to inflammation can include any incremental step that results in less inflammation, such as less redness, temperature, swelling, and/or pain. Additionally, the use of the term can include from any minimal decrease to absolute abolishment of a physiological process or effect.

As used herein, “treat,” “treatment,” or “treating” refers to the process or result of giving medical aid to a subject, where the medical aid can counteract a malady, a symptom thereof, or other related adverse physiological manifestation. Additionally, these terms can refer to the administration or application of remedies to a patient or for a disease or injury; such as a medicine or a therapy. Accordingly, the substance or remedy so applied, such as the process of providing procedures or applications, are intended to relieve illness, injury or inflammation. Additionally, the term can be used for the procedure of preemptively acting to prevent the malady, a symptom thereof, or other related adverse physiological manifestation. As such, a treatment can be administered prior to the subject experiencing any symptoms so that the symptoms are not manifested in the subject.

As used herein, “carrier” or “inert carrier” refers to typical compounds or compositions used to carry active ingredients, such as polymeric carriers, liquid carriers, or other carrier vehicles with which a bioactive agent, such as a DHEA congener and/or other anti-inflammatory agents, may be combined to achieve a specific dosage form. As a generally principle, carriers do not substantially react with the bioactive agent in a manner which substantially degrades or otherwise adversely affects the bioactive agent or its therapeutic potential.

As used herein, “subject” refers to an animal, such as a mammal, that may benefit from the administration of an inflammation reducing drug, a combination of drugs, or a formulation; or from a method for achieving reduced inflammation recited herein. Most often, the subject will be a human.

The term “dehydroepiandrosterone congener” or “DHEA congener” includes dehydroepiandrosterone (a.k.a. DHEA and (3β)-3-hydroxyandrost-5-en-17-one), derivatives of DHEA, metabolites of DHEA, metabolites of DHEA derivatives, salts of DHEA, salts of DHEA derivatives, etc. DHEA, generally, is a weak androgen that serves as the primary precursor in the biosynthesis of both androgens and estrogens. Typically, a DHEA congener used in accordance with embodiments of the present invention is in a pharmaceutically acceptable form.

As used herein, “mc” or “micro” when used in combination with a unit of measurement denotes the standard unit to be divided by one million, or multiplied by 1×10⁶. Accordingly, the prefix “micro,” which is well known by one or ordinary skill in the art can be referred herein by the abbreviation “mc.”

As used herein, “mg/kg” or any other mass unit divided by another mass unit when used to describe a drug dose or dosing regimen denotes the mass of drug delivered per mass of the subject being administered the drug. Such use of units when referring to pharmaceuticals and their associated doses is well known to one of ordinary skill in the art.

As used herein, “mg/m²” or any other mass unit divided by an area unit when used to describe a drug dose or dosing regimen denotes the mass of the drug delivered per surface area of the subject being administered the drug. The use of mass of drug per surface area of subject when referring to pharmaceuticals and their associated doses is well known to one of ordinary skill in the art.

As used herein, “enhance” or “enhancing” of an anti-inflammatory response refers to the interaction of two or more active agents or drugs so that their combined physiological effect is greater than the individual effect of either active agent when administered alone at the same dosage. “Synergism” or “synergistic effect” refers to an anti-inflammatory response where the interaction of two or more active agents or drugs provides a combined physiological effect that is greater than the additive effect of both active agents.

The term “anti-TNF-α agent” refers to compositions or compounds that act to inhibit the normal function tumor necrosis factor alpha (TNFα), which are important cytokines involved in systemic inflammation and the acute phase response.

The term “about” when referring to a numerical value or range is intended to encompass the values resulting from experimental error that can occur when taking measurements.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 wt % to about 20 wt % should be interpreted to include not only the explicitly recited concentration limits of 1 wt % to about 20 wt %, but also to include individual concentrations such as 2 wt %, 3 wt %, 4 wt %, and sub-ranges such as 5 wt % to 15 wt %, 10 wt % to 20 wt %, etc.

A) Dehydroepiandrosterone Congeners

As stated, a DHEA congener includes DHEA (3β)-3-hydroxyandrost-5-en-17-one), derivatives of DHEA, metabolites of DHEA, metabolites of DHEA derivatives, salts of DHEA, salts of DHEA derivatives, etc. Typically, a DHEA congener used in accordance with embodiments of the present invention is in a pharmaceutically acceptable form. Examples of DHEA congeners include, but are not limited to, compounds having the general formula I, and their metabolites and pharmaceutically acceptable salts thereof: 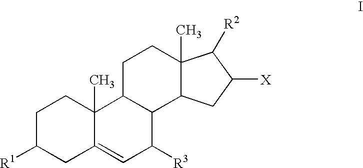
wherein
X is H or halogen;
R¹, R² and R³ are independently ═O, —OH, —SH, H, halogen, pharmaceutically acceptable esters, pharmaceutically acceptable thioesters, pharmaceutically acceptable ethers, pharmaceutically acceptable thioethers, pharmaceutically acceptable inorganic esters, pharmaceutically acceptable monosaccharides, disaccharides or oligosaccharides, spirooxiranes, spirothiranes, —OSO₂R⁴ or —OPOR⁴R⁵;
R⁴ and R⁵ are independently —OH, pharmaceutically acceptable esters or pharmaceutically acceptable ethers.

Suitable metabolites of DHEA include, but are not limited to, dehydroepiandrosterone sulfate, 16α-hydroxydehydroepiandrosterone, 16α-hydroxyandrost-4-ene-3,17dione, androst-4-ene-3,17 dione, 7α-hydroxyandrostenedione, 7α-hydroxytestosterone.

Further examples of DHEA congeners, include but are not limited to, compounds having the general formulas II and III, and their metabolites and pharmaceutically acceptable salts thereof: 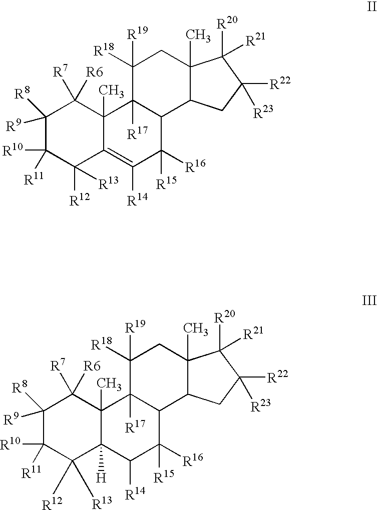
wherein
R⁶, R⁷, R⁸, R⁹, R¹¹, R¹², R¹³, R¹⁴, R¹⁵, R¹⁶, R¹⁷, R¹⁸, R¹⁹ and R²⁴ are independently H, —OH, halogen, C₁₋₁₀ alkyl or C₁₋₁₀ alkoxy;
R¹⁰ is H, —OH, halogen, C₁₋₁₀ alkyl, or C₁₋₁₀ alkoxy;
R²⁰ is (1) H, halogen, C₁₋₁₀ alkyl or C₁₋₁₀ alkoxy when R²¹ is —C(O)OR²⁵ or
(2) H, halogen, OH or C₁₋₁₀ alkyl when R²¹ is H, halogen, OH or C₁₋₁₀ alkyl or
(3) H, halogen, C₁₋₁₀ alkyl, C₁₋₁₀ alkenyl, C₁₋₁₀ alkynyl, formyl, C₁₋₁₀ alkanoyl or epoxy when R²¹ is OH; or
R²⁰ and R²¹ taken together are ═O;
R²² and R²³ are independently (1) H, —OH, halogen, C₁₋₁₀ alkyl or C₁₋₁₀ alkoxy when R²¹ is H, OH, halogen, C₁₋₁₀ alkyl or —C(O)OR²⁵ or (2) H, (C₁₋₁₀ alkyl)ₙamino, (C₁₋₁₀ alkyl)ₙamino-C₁₋₁₀ alkyl, C₁₋₁₀ alkoxy, hydroxy-C₁₋₁₀ alkyl, C₁₋₁₀ alkoxy-C₁₋₁₀ alkyl, (halogen)ₘ—C₁₋₁₀ alkyl, C₁₋₁₀ alkanoyl, formyl, C₁₋₁₀ carbalkoxy or C₁₋₁₀ alkanoyloxy when R²⁰ and R²¹ taken together are ═O; or
R²² and R²³ taken together are ═O or taken together with the carbon to which they are attached form a 3-6 member ring containing 0 or 1 oxygen atom; or
R²⁰ and R²² taken together with the carbons to which they are attached form an epoxide ring;
R²⁵ is H, (halogen)ₘ—C₁₋₁₀ alkyl or C₁₋₁₀ alkyl;
n is 0, 1 or 2;
m is 1, 2 or 3; and
physiologically acceptable salts thereof, with the provisos that
(a) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ and R²¹ taken together are ═O; and
(b) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ is H and R²¹ is H, OH or halogen.

The compounds represented by the general formula I exist in many stereoisomers and the formula is intended to encompass the various stereoisomers. Examples of suitable DHEA congeners of Formula I include compounds in which:
(1) R² is ═O, R³ and X are each H and R¹ is ═O, —OH, pharmaceutically acceptable esters thereof, pharmaceutically acceptable ethers thereof or pharmaceutically acceptable salts;
(2) R² is ═O, R³ is H, X is halogen and R¹ is ═O, —OH, pharmaceutically acceptable esters thereof, pharmaceutically acceptable ethers thereof or pharmaceutically acceptable salts;
(3) R² is ═O, R³ and X are each H and R¹ is —SH, pharmaceutically acceptable thioesters thereof, pharmaceutically acceptable thioethers thereof or pharmaceutically acceptable salts;
(4) R² is ═O, R³ is H, X is halogen and R¹ is —SH, pharmaceutically acceptable thioesters thereof, pharmaceutically acceptable thioethers thereof or pharmaceutically acceptable salts;
(5) R² is ═O, X is H and R¹ and R³ are independently ═O, —OH, pharmaceutically acceptable esters thereof, pharmaceutically acceptable ethers thereof or pharmaceutically acceptable salts;
(6) R² is ═O, X is halogen and R¹ and R³ are independently ═O, —OH, pharmaceutically acceptable esters thereof, pharmaceutically acceptable ethers thereof or pharmaceutically acceptable salts;
(7) R² is ═O, X is H and R¹ and R³ are independently —SH, pharmaceutically acceptable thioesters thereof, pharmaceutically acceptable thioethers thereof or pharmaceutically acceptable salts;
(8) R² is ═O, X is halogen and R¹ and R³ are independently —SH, pharmaceutically acceptable thioesters thereof, pharmaceutically acceptable thioethers thereof or pharmaceutically acceptable salts;
(9) R² is —OH, R³ and X are each H and R¹ is ═O, —OH, pharmaceutically acceptable esters thereof, pharmaceutically acceptable ethers thereof or pharmaceutically acceptable salts;
(10) R² is —OH, R³ is H, X is halogen and R¹ is ═O, —OH, pharmaceutically acceptable esters thereof, pharmaceutically acceptable ethers thereof or pharmaceutically acceptable salts;
(11) R² is —OH, R³ and X are each H and R¹ is —SH, pharmaceutically acceptable thioesters thereof, pharmaceutically acceptable thioethers thereof or pharmaceutically acceptable salts;
(12) R² is —OH, R³ is H, X is halogen and R¹ is —SH, pharmaceutically acceptable thioesters thereof, pharmaceutically acceptable thioethers thereof or pharmaceutically acceptable salts;
(13) R² is —OH, X is H and R¹ and R³ are independently ═O, —OH, pharmaceutically acceptable esters thereof, pharmaceutically acceptable ethers thereof or pharmaceutically acceptable salts;
(14) R² is —OH, X is halogen and R¹ and R³ are independently ═O, —OH, pharmaceutically acceptable esters thereof, pharmaceutically acceptable ethers thereof or pharmaceutically acceptable salts;
(15) R² is —OH, X is H and R¹ and R³ are independently —SH, pharmaceutically acceptable thioesters thereof, pharmaceutically acceptable thioethers thereof or pharmaceutically acceptable salts;
(16) R² is —OH, X is halogen and R¹ and R³ are independently —SH, pharmaceutically acceptable thioesters thereof, pharmaceutically acceptable thioethers thereof or pharmaceutically acceptable salts;
(17) R² is —SH, R³ and X are each H and R¹ is ═O, —OH, pharmaceutically acceptable esters thereof, pharmaceutically acceptable ethers thereof or pharmaceutically acceptable salts;
(18) R² is —SH, R³ is H, X is halogen and R¹ is ═O, —OH, pharmaceutically acceptable esters thereof, pharmaceutically acceptable ethers thereof or pharmaceutically acceptable salts;
(19) R² is —SH, R³ and X are each H and R¹ is —SH, pharmaceutically acceptable thioesters thereof, pharmaceutically acceptable thioethers thereof or pharmaceutically acceptable salts;
(20) R² is —SH, R³ is H, X is halogen and R¹ is —SH, pharmaceutically acceptable thioesters thereof, pharmaceutically acceptable thioethers thereof or pharmaceutically acceptable salts;
(21) R² is —SH, X is H and R¹ and R³ are independently ═O, —OH, pharmaceutically acceptable esters thereof, pharmaceutically acceptable ethers thereof or pharmaceutically acceptable salts;
(22) R² is —SH, X is halogen and R¹ and R³ are independently ═O, —OH, pharmaceutically acceptable esters thereof, pharmaceutically acceptable ethers thereof or pharmaceutically acceptable salts;
(23) R² is —SH, X is H and R¹ and R³ are independently —SH, pharmaceutically acceptable thioesters thereof, pharmaceutically acceptable thioethers thereof or pharmaceutically acceptable salts;
(24) R² is —SH, X is halogen and R¹ and R³ are independently —SH, pharmaceutically acceptable thioesters thereof, pharmaceutically acceptable thioethers thereof or pharmaceutically acceptable salts;
(25) X is H and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;
(26) X is halogen and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;
(27) X is H, R¹ is ═O or —OH, and R² and R³ are independently ═O, —OH, pharmaceutically acceptable inorganic esters thereof or pharmaceutically acceptable salts, wherein at least one of R² and R³ is an inorganic ester; and/or
(28) X is halogen R¹ is ═O or —OH, and R² and R³ are independently ═O, —OH, pharmaceutically acceptable inorganic esters thereof or pharmaceutically acceptable salts, wherein at least one of R² and R³ is an inorganic ester.

Pharmaceutically acceptable esters or thioesters include, but are not limited to, esters or thioesters of the formula —OOCR or —SOCR, wherein R is a pharmaceutically acceptable alkyl, alkenyl, aryl, alkylaryl, arylalkyl, sphingosine or substituted sphingolipid groups, such as propionate, enanthate, cypionate, succinate, decanoate and phenylpropionate esters.

Pharmaceutically acceptable ethers or thioethers include, but are not limited to, ethers or thioethers of the formula —OR or —SR, wherein R is as defined above or enol, or —OR is an unsubstituted or substituted spirooxirane or —SR is a spirothiane.

Suitable sugar residues can include, but are not limited to monosaccharides, disaccharides, and oligosaccharides, such as a glucuronate.

Pharmaceutically acceptable inorganic esters include, but are not limited to, esters of the formula —OSO₂R⁴ or —OPOR⁴R⁵, wherein R⁴ and R⁵ are independently —OH, pharmaceutically acceptable esters, pharmaceutically acceptable ethers or pharmaceutically acceptable salts.

Some DHEA congeners, such as the compounds of general formulas I, II, and III, can be synthesized as described in U.S. Pat. Nos. 4,898,694; 5,001,119; 5,028,631; and 5,175,154, which are all incorporated herein by reference. The compounds represented by the general formulas II and III exist in many stereoisomers and these formulas are intended to encompass the various stereoisomers. Examples of representative compounds, which fall within the scope of general formulas II and III, include the following: 5α-androstan-17-one; 16α-fluoro-5α-androstan-17-one; 3β-methyl-5α-androsten-17-one; 16β-fluoro-5α-androstan-17-one; 17β-bromo-5-androsten-16-one; 17β-fluoro-3β-methyl-5-androsten-16-one; 17α-fluoro-5α-androstan-16-one; 3β-hydroxy-5-androsten-17-one; 17α-methyl-5α-androstan-16-one; 16α-methyl-5-androsten-17-one; 3β,16α-dimethyl-5-androsten-17-one; 3β,17α-dimethyl-5-androsten-16-one; 16α-hydroxy-5-androsten-17-one; 16β-fluoro-16β-methyl-5-androsten-17-one; 16α-methyl-5α-androstan-17-one; 16-dimethylaminomethyl-5α-androstan-17-one; 16β-methoxy-5-androsten-17-one; 16α-fluoromethyl-5-androsten-17-one; 16-methylene-5-androsten-17-one; 16-cyclopropyl-5α-androstan-17-one; 16-cyclobutyl-5-androsten-17-one; 16-hydroxymethylene-5-androsten-17-one; 3α-bromo-16α-methoxy-5-androsten-17-one; 16-oxymethylene-5-androsten-17-one; 3β-methyl-16ξ-trifluoromethyl-5α-androstan-17-one; 16-carbomethoxy-5-androsten-17-one; 3β-methyl-16β-methoxy-5α-androstan-17-one; 3β-hydroxy-16α-dimethylamino-5-androsten-17-one; 17α-methyl-5-androsten-17β-ol; 17α-ethynyl-5α-androstan-17β-ol; 17β-formyl-5α-androstan-17β-ol; 20,21-epoxy-5α-pregnan-17α-ol; 3β-hydroxy-20,21-epoxy-5α-pregnan-17α-ol; 16α-fluoro-17α-ethenyl-5-androsten-17β-ol; 16α-hydroxy-5-androsten-17α-ol; 16α-methyl-5α-androstan-17α-ol; 16α-methyl-16β-fluoro-5α-androstan-17α-ol; 16α-methyl-16β-fluoro-3-hydroxy-5-androsten-17α-ol; 3β,16β-dimethyl-5-androsten-17β-ol; 3β,16,16-trimethyl-5-androsten-17β-ol; 3β,16,16-trimethyl-5-androsten-17-one; 3β-hydroxy-4α-methyl-5-androsten-17α-ol; 3β-hydroxy-4α-methyl-5-androsten-17-one; 3α-hydroxy-1α-methyl-5-androsten-17-one; 3α-ethoxy-5α-androstan-17β-ol; 5α-pregnan-20-one; 3β-methyl-5α-pregnan-20-one; 16α-methyl-5-pregnen-20-one; 16α-methyl-3β-hydroxy-5-pregnen-20-one; 17α-fluoro-5-pregnen-20-one; 21-fluoro-5α-pregnan-20-one; 17α-methyl-5-pregnen-20-one; 20-acetoxy-cis-17(20)-5α-pregnene; and 3α-methyl-16,17-epoxy-5-pregnen-20-one, for example.

In one aspect of the present invention, a DHEA congener and a second anti-inflammatory agent can be co-administered to a subject in an amount that results in a therapeutic effect, thereby aiding in treating and/or preventing inflammation in a subject. The dose of the DHEA congener administered is selected to achieve DHEA or DHEA equivalent blood levels greater than normal endogenous DHEA blood levels. Normal endogenous blood levels of DHEA can be less than 20 ng/mL. Accordingly, peak blood levels of DHEA or DHEA equivalent can be greater than about 20 ng/mL, or as desired for a specific therapeutic effect. In another aspect, suitable doses that are selected to achieve a peak blood level of DHEA or DHEA equivalent can be in the range from about 30 ng/mL to about 100 mg/mL, or in the range from about 50 ng/mL to about 10 mg/mL. Additionally, the doses administered to a subject can be in an amount to achieve DHEA blood levels in the subject from about 100 ng/mL to about 1 mg/mL, from 100 ng/mL to about 100 μg/mL, and/or from about 100 ng/mL to about 10 μg/mL.

In accordance with the methods of the present invention, a DHEA congener can be administered as a part of a regimen to aid in the reduction of subchronic to chronic inflammation as well as acute inflammation. In one aspect, a DHEA congener can be administered in a dosing regimen that includes providing from about 1 mg to about 200 mg per day of the DHEA congener to a subject to aid in reducing subchronic to chronic inflammation. In another aspect, a DHEA congener can be administered in a dosing regimen that includes providing from about 10 mg to about 3600 mg of the DHEA congener to a subject to aid in reducing acute inflammation. In still a further aspect of the present invention, a DHEA congener can be administered in a dosing regimen to aid in preventing the onset of inflammation, which includes providing from about 10 mg to about 3600 mg per day of the DHEA congener to a subject not yet experiencing observable inflammation. These dosages can be administered once a day, or at smaller dosages throughout the day.

It is to be noted that the present invention is related to co-administration of DHEA congeners with other anti-inflammatory agents. This co-administration can result in an enhancing effect, or even a synergistic effect. Each dosage for the “second anti-inflammatory agent” will be provided below individually. However, though second anti-inflammatory agent dosages are provided individually, it is understood that the DHEA congener can be co-administered with the second anti-inflammatory agents described below within the DHEA congener dosage ranges provided above. Further, it is understood that other functional ranges outside of the ranges provided within the present disclosure, though not specifically mentioned, are included to the extent that such dosage ranges are functional.

B) Anti-Inflammatory Agents for Administration with DHEA Congener

As described, DHEA congeners can be used in effective dosing regimens for reducing inflammation. As such, DHEA can be co-administered with any of a variety of anti-inflammatory agents, referred to generally as “second anti-inflammatory agents,” in accordance with methods of the present invention to more effectively reduce inflammation or treat inflammatory producing diseases. Accordingly, the DHEA congener and the second anti-inflammatory agent can each be administered at a therapeutically effective dosage, such that the inflammation can be reduced more than if each composition were administered alone. Examples of conditions that would benefit from the methods of the present invention include rheumatoid arthritis, asthma, inflammatory bowel disease, Crohn's disease, ulcerative colitis, multiple sclerosis, chronic obstructive pulmonary disease (COPD), allergies and associated allergic reactions, allergic rhinitis (hay fever), rheumatic fever, heart disease, bleeding disorders such as thromboycytopenia, kidney inflammation, lupus, atopic dermatitis, tissue necrosis, tuberculosis, chronic cholecystitis, brohchiectasis, Hashimoto's thyroiditis, pneumoconiosis such as silicosis, pelvic inflammatory disease (PID), chronic sarcoidosis, and pancreatitis, as well as other maladies that end with the suffix “itis.”

A brief summary of several exemplary inflammatory diseases are described herein, along with specific anti-inflammatory agents that can be co-administered with DHEA to achieve an anti-inflammatory therapeutic effect. In accordance with these exemplary inflammatory conditions and exemplary effective anti-inflammatory agents for use in accordance with embodiments of the present invention, several specific dosage examples, dosage timing, dosage periods, administration routes, etc, are provided. However, it is to be understood that this information is provided for exemplary purposes only. These and other administration considerations can be varied by one skilled in the art (for administration with DHEA congener) in order to achieve a therapeutic effect. In other words, as one skilled in the art understands, dosages, timing of administration, length of administration, administration routes, drug selection, etc., should considered on a case by case basis. As an example of the variability that can exist, when considering the dosages provided herein with respect to the second anti-inflammatory agent, the dosages can be administered at from about 20% to about 500% the dosages provided, for example. As another example, several drugs are listed as being effective for treating specific diseases or conditions by administration with DHEA congener. These lists are not considered to be exhaustive, and to the extent that a second anti-inflammatory agent can be administered with DHEA congener to enhance an anti-inflammatory response, that composition can be administered in accordance with embodiments of the present invention. Further, specific second anti-inflammatory agents are associated with specific diseases. This is for exemplary purposes only, as any second drug can be used to treat any inflammatory disease when co-administered with DHEA congeners, limited only by functionality. When referring to dosages, typically, it is understood that the dosage amounts are for oral administration unless stated otherwise, or unless the context dictates otherwise.

i) Rheumatoid Arthritis

Rheumatoid arthritis can be a chronic disease marked by stiffness and inflammation of the joints, weakness, loss of mobility, and deformity, where subjects suffering therefrom typically experience chronic flare-ups of inflammation. It is also known as an autoimmune disease because of its association with inappropriate immunological responses by the body against healthy joint tissue that can cause inflammation and subsequent joint damage.

A method of reducing inflammation in a subject such as those including the co-administration of a DHEA congener and a second anti-inflammatory agent to the subject can be used in treating and/or preventing rheumatoid arthritis and/or the adverse symptoms thereof. Such second anti-inflammatory agents that can be delivered with a DHEA congener for the treatment of rheumatoid arthritis can include phenylbutazone, oxyohenbutazone, antipyrine, aminopyrine, aurothioglucose, gold, sodium thiomalate, auranofin, cyclosporine, azatioprine, methotrexate, glucocoriticoids, penicillamine, hydoxychloroquine, etodolac, mefenamic acid, meclofenamate sodium, totmetin, ketorolac, diclofenac, ibuprofen, leflunomide, naproxen, naproxen sodium, fenoprofen, ketoprofen, fluribiprofen, oxaprozin, celecoxib, rofecoxib, apazone, N-(4-nitro-2-phenoxyphenyl)-methanesulfonamide (nimesulide), monoclonal antibodies such as infliximab, Vioxx (rofecoxib), acetylsalicylic acid, and combinations thereof. In certain specific embodiments, the following second anti-inflammatory agent dosage amounts can be co-administered with a DHEA congener for treating rheumatoid arthritis.

In one aspect of the present invention, leflunomide, or N-(4′-trifluoromethylphenyl)-5-methyllisoxazole-4-carboxamide, can be co-ad ministered with a DHEA congener at a loading dose of about 100 mg per 3 days. In another aspect, leflunomide and can be administered as a maintenance dose of about 10 mg to about 20 mg per day.

Additionally, an aspect of the present invention for treating rheumatoid arthritis provides for the co-administration of the second anti-inflammatory agent methotrexate (N-[4-[2,4-diamino-6-pteridinyl)methyl-amino]benzoyl]-L-glutamic acid), which can be administered at a dosage of about 7.5 mg per week to about 15 mg per week.

In another aspect of treating rheumatoid arthritis, the second anti-inflammatory agent Vioxx® (rofecoxib or 4-[4-(methylsulfonyl)phenyl]-3-phenyl-2(5H)-furanone), can be co-administered at a dosage of about 25 mg/day.

In still another aspect, the second anti-inflammatory agent Remicade (anti-TNF-α or infliximab), which is a chimeric anti-TNF-α (IgG1) monoclonal antibody, can be co-administered at initial doses of about 3 mg/kg intravenously on the first day of the regimen, and then at about 2 weeks, and again at about 6 weeks. In yet another aspect, maintenance doses can be administered in a dose of about 3 mg/kg at about every 8 weeks after the initial doses.

In a further aspect of treating rheumatoid arthritis, the second anti-inflammatory agent Celebrex® (celecoxib or 4-[5-(4-methylphenyl)-3-(trifluoromethyl)-1H-pyrazol-1-y] benzenesulfonamide), can be co-administered at doses ranging from about 10 mg twice a day to about 200 mg twice a day.

ii) Asthma

Asthma or any asthmatic condition can include the physiological consequences that occur when a subject has an increased responsiveness of the tracheobronchial tree when exposed to various stimuli, which results in paroxysmal constriction of the bronchial airways. Accordingly, asthma can include the chronic inflammation of the bronchial tubes (airways) that causes swelling and narrowing (constriction) of the airways.

A method of reducing inflammation in a subject such as those including the co-administration of a DHEA congener and a second anti-inflammatory agent to the subject can be used in treating and/or preventing asthma, asthmatic conditions and reactions, and/or adverse symptoms thereof. Such second anti-inflammatory agents that can be delivered with a DHEA congener for the treatment of asthma can include 5-lipooxygenase, docebenone, ICI-D2318, MK-0591, MK-886, Piripost, zileuton, corticosteroids, beclomethasone dipropionate, budesonide, flunisolide, triamcinolone acetonide, prednisone or prednisolone, cromolyn, nedocromil, albuterol, epinephrine, ipratropium, metaproterenol, terbutaline, and combinations thereof. In certain specific embodiments, the following second anti-inflammatory agent dosage amounts can be co-administered with a DHEA congener for treating asthma.

In one aspect, the second anti-inflammatory agent Prelone (prednisolone or 11β,17,21-Trihydrox-pregna-1,4 diene-3,20 dione) can be co-administered at a dose of about 5 mcg per day to about 60 mcg per day.

In another aspect related to treating asthma, the second anti-inflammatory agent Combivent (ipratropium bromide in conjunction albuterol sulfate), which is also known as 8-azinabicyclo[3.2.1]octane, 3-(3-hydroxy-1-oxo-2-phenylpropoxy)-8-methyl-8-(1-methylethyl)-bromide, monohydrate (endo, syn)-, (+/−)-, in conjunction with 1,3-benzene-dimethanol, α′-[[(1,1-dimethylethyl)amino] methyl]-4-hydroxy, sulfate (2:1)(salt), (+/−)), respectively, can be used to treat and/or prevent asthma. Along with a DHEA congener, the combination of ipratropium bromide and albuterol sufphate can also be co-administered at a dosage of about 2 to about 4 inhalations per day, where the regimen should not exceed 12 inhalations in one day. In another aspect of treating asthma, the dosage amount of ipratropium bromide can be about 18 mcg, and the dosage amount of albuterol sulfate can be about 03 mcg, which can further be about 90 mcg of the albuterol base.

iii) Inflammatory Bowel Disease

Inflammatory Bowel Disease (IBD) can be diagnosed by the observation of chronic intestinal inflammation, which can result in symptoms including diarrhea, bleeding, abdominal pain, fever, joint pain, and/or weight loss. However, these symptoms can range from mild to severe, which can be exemplified by the gradual development from minor to severe sensations, or by sudden and severe intensity. IBD can be manifested in various forms, which includes the two major forms of Crohn's disease and ulcerative colitis. While these two forms of IBD can appear very similar with regard to their symptoms, ulcerative colitis can primarily involve inflammation of the colon and rectum. On the other hand Crohn's disease can impact the upper intestinal digestive tract.

Specifically, Crohn's disease, also known as regional enteritis, granulomatous ileitis, and/or ileocolitis, can be a nonspecific chronic transmural inflammatory disease that most commonly affects the distal ileum and colon but may occur in any part of the GI tract. Accordingly, this can cause decreased absorption of food, which in turn can cause chronic vitamin and nutrient deficiencies. Neither the fundamental cause of, nor the cure for, Crohn's disease is known. Evidence suggests that a genetic predisposition can lead to an unregulated intestinal inflammatory response to an environmental, dietary, or infectious agent. Cigarette smoking seems to contribute to the development or exacerbation of Crohn's disease. The earliest mucosal lesion of Crohn's disease can be crypt injury in the form of inflammation (cryptitis) and crypt abscesses, which can further progress to tiny focal aphthoid ulcers. The transmural spread of inflammation can lead to lymphedema and bowel wall thickening, which may eventually result in extensive fibrosis. In conjunction with the administration of DHEA congeners, cramps and diarrhea can be more effectively relieved by oral administration of anticholinergics. Additionally, DHEA congeners along with sulfasalazine and mesalamine (5-aminosalicylic acid) can also benefit patients with mild to moderate colitis and ileocolitis, and can have some efficacy in ileitis as well as maintaining remission. Also in conjunction with DHEA congeners, corticosteroids can aid in treating the acute stages of Crohn's disease by dramatically reducing fever and diarrhea, relieving abdominal pain and tenderness, and improving the appetite and sense of well-being. Additionally, DHEA congeners along with azathioprine and 6-mercaptopurine can also be used to aid in the long-term therapy for Crohn's disease. Additionally, DHEA congeners and high-doses of cyclosporine can aid in treating inflammatory and fistulous diseases, but its long-term use can be contraindicated by multiple toxicities. Also, DHEA congeners and infliximab, a monoclonal antibody that inhibits tumor necrosis factor, can be administered for moderate to severe Crohn's disease (especially fistulous disease) refractory to other treatments, however, long-term efficacy and side effects remain to be determined when administering infliximab alone or with other drugs.

Ulcerative colitis is a disease that can cause inflammation and sores in the lining of the large intestine, which leads to the ulcers. The inflammation can occur in the rectum and in lower part of the colon. Additionally, it can cause ulcers in the entire colon, but usually does not affect the small intestine. Also, ulcerative colitis has been referred to as colitis or proctitis. These ulcers can arise where the inflammation has killed cells lining the colon, which can bleed and produce pus. The inflammation can cause intestinal and/or colon ultra motility, which frequently results in diarrhea.

A method of reducing inflammation in a subject such as those including the co-administration of a DHEA congener and a second anti-inflammatory agent to the subject can be used in treating and/or preventing various forms of IBD. More specifically, the DHEA congener can be co-administered with the second anti-inflammatory agent to treat and/or prevent ulcerative colitis and/or Crohn's disease. In addition to the drugs discussed above, second anti-inflammatory agents that can be delivered with a DHEA congener for the treatment of associated IBDs include glucocortioid, hydrocortisone, prednisone, prednisolone, 5-aminosalicyclic acid, sodium azodisalicylate, mesalamine, salicylazosulfapyridine, sulfasalzine, azaline, Azulfidine, diflunisal, olsalazine (Dipentum) and balsalazide (Colazal), anticholinergics, diphenoxylate, loperamide, deodorized opium tincture, codeine, budesonide, infliximab, daclizumab, and combinations thereof. In certain specific embodiments, the following second anti-inflammatory agent dosage amounts that can be co-administered with a DHEA congener for treating IBDs include the following.

In one aspect of the present invention, the second anti-inflammatory agent Entocort (budesonide or (RS)-11β,16α,17,21-tetrahydroxypregna-1,4-diene-3,20-dione cyclic 16,17-acetal with butyraldehyde), can be co-administered at a dosage of about 9 mg per day. In another aspect, the dosing regimen can be administered for about 8 weeks.

In another aspect of treating an IBD, the second anti-inflammatory agent Prelone (prednisolone or 11β,17,21-Trihydrox-pregna-1,4 diene-3,20 dione) can be co-administered at a dosage of about 5 mcg per day to about 60 mcg per day.

In another aspect of treating an IBD, the second anti-inflammatory agent Remicade (infliximab or anti-TNF-α), which is a chimeric anti-TNF-α (IgG1) monoclonal antibody, can be co-administered at an initial doses of 5 mg/kg to about 10 mg/kg intravenously on the first day of the regimen, and at about 2 weeks, and again at about 6 weeks. In still another aspect, at about every 8 weeks a maintenance dose can be administered of about 5 mg/kg to about 10 mg/kg. In yet another aspect, when there has not been a response to the dosing regimen, a non-response dosage of about 10 mg/kg can be administered.

In still another aspect of treating IBD, the second anti-inflammatory agent Azulfidine (enteric coated sulfasalazine or 5-([p-(2-pyridylsulfamoyl)pheny-]azo) salicylic acid) can be co-administered at a dosage of about 2 grams per day for adults. In another aspect, a dosing regimen for children can include administering a total of about 30 mg/kg of body weight per day, which is divided into about 4 equal doses.

In yet another aspect of treating IBD, the second anti-inflammatory agent Dipentum (olsalazine sodium or disodium 3,3′-azobis (6-hydroxybenzoate)), which is a compound that can be effectively bioconverted to 5-aminosalicylic acid (5-ASA), can be co-administered at a dosage of about 1.0 gram per day.

In a further aspect of treating IBD, the second anti-inflammatory agent Zenapax (daclizumab), which is a humanized IgG1 monoclonal antibody directed against the p55 alpha (CD25) subunit of the IL-2 receptor, can be co-administered at a dosage of about 1.0 mg/kg at about every 2 weeks. In another aspect, the regimen can include a total of about 5 doses.

iv) Multiple Sclerosis

Multiple sclerosis (MS) can be a chronic and potentially debilitating malady that affects the brain and/or spinal cord (central nervous system). Research has indicated that MS is likely an autoimmune disease because the immune system responds by attacking the body similarly to how the immune system attacks a foreign substance. The response can include antibodies and white blood cells being directed to attack proteins in the myelin sheath surrounding the nerves in brain and spinal cord. This can cause inflammation and injury to the sheath and ultimately to nerves inside the myelin sheath, which can cause scarring (sclerosis) to multiple areas. The damage to the myelin sheath and/or the nerves inside can slow or block many nerve signals, and may affect muscle coordination, visual sensation among others.

Methods of reducing inflammation in a subject such as those including the co-administration of a DHEA congener and a second anti-inflammatory agent to the subject can be used in treating and/or preventing multiple sclerosis. Such second anti-inflammatory agents that can be delivered with a DHEA congener for the treatment of multiple sclerosis can include methylprednisone, prednisone, propantheline bromide, oxybutynin, tolterodine tartrate (Detrol), corticosteroids, interferon β-1b (Betaseron), interferon β-1a (Avonex), High-dose interferon β-1a (Rebif), glatiramer (Copaxone), and mitoxantrone (Novantrone), and combinations thereof. In certain specific embodiments, the following second anti-inflammatory agent dosage amounts can be co-administered with a DHEA congener for treating multiple sclerosis.

In one aspect of the present invention, the second anti-inflammatory agent Avonex (interferon β-1a), which is a recombinant IFN-beta protein with human albumin, sodium chloride, and sodium phosphate, can be co-administered at a dosage of about 30 mcg per week. In another aspect, the dose can be given intra-muscularly.

In another aspect of treating multiple sclerosis, the second anti-inflammatory agent Novantrone (mitoxantrone hydrochloride or 1,4-dihydroxy-5,8-bis[[2-hydroxyethyl) amino] ethyl]amino]-9,10-anthracenedione dihydrochloride), can be co-administered at a dosage of about 12 mg/m². In another aspect, the dosage can be administered intravenously. In yet another aspect, the dosage can be administered about every 3 months. In still a further aspect, the summation of all dosages should not exceed a lifetime total dose of about 140 mg/m².

In yet another aspect of treating multiple sclerosis, the second anti-inflammatory agent Copaxone (glatriramer acetate), which can be a L-glutamic acid polymer with L-alanine, L-tyrosine, and L-lysine, can be co-administered at a dosage of about 20 mg per day. In another aspect, the administration route can be by subcutaneous injection.

In still another aspect of treating multiple sclerosis, the second anti-inflammatory agent Detrol (tolterodine tartrate or R-2-[3-[bis(1-methylethyl)-amino]-1-phenylpropyl]-4-methylphenol [R-(R,R)]-2,3-dihydroxybutanedioate (1:1) salt) can be co-administered at a dosage range of about 2 mg per day to about 4 mg per day.

v) Chronic Obstructive Pulmonary Disease

Chronic obstructive pulmonary disease (COPD) is a lung disease in which the pulmonary airways and/or lung is damaged, which can make it difficult to breathe. Accordingly, during COPD, the tubes that carry air in and out of the lungs can be partly obstructed, and it can be difficult getting air in and out during respiration. Breathing in different kinds of lung irritants, like pollution, dust, chemicals, or cigarette smoke over a period of time can cause or contribute to the development and agitation of COPD.

As such, methods of reducing inflammation in a subject such as those including the co-administration of a DHEA congener and a second anti-inflammatory agent to the subject can be used in treating and/or preventing COPD. Such second anti-inflammatory agents that can be delivered with a DHEA congener for the treatment of COPD can include P2-adrenergic agonists, corticosteroids, beclomethasone dipropionate, budesonide, flunisolide, triamcinolone acetonide, prednisone, prednisolone, beclomethasone dipropionate, triamcinolone acetonide, flunisolide, budesonide, albuterol, epinephrine, ipratropium, metaproterenol, terbutaline, and combinations thereof. In certain specific embodiments, the following second anti-inflammatory agent dosage amounts can be co-administered with a DHEA congener for treating COPD.

In one aspect of the present invention, the second anti-inflammatory agent Pulmicort Turbuhaler (budesonide inhalation powder or (RS)-11β,16α,17,21-tetrahydroxypregna-1,4-diene-3,20-dione cyclic 16,17-acetal with butyraldehyde) can be co-administered at a dosage range of about 200 mcg to about 800 mcg twice a day.

In another aspect of treating COPD, the second anti-inflammatory agent Beconase (beclomethasone dipropionate or 9-chloro-11β,17,21-trihydroxy-16α-methylpregna-1,4-diene-3,20-dione 17,21-dipropionate) can be co-administered at a dosage range of about 168 to about 336 mcg per day.

In yet another aspect of treating COPD, the second anti-inflammatory agent Alupent (metaproterenol sulfate or 1-(3,5-dihydroxyphenyl)-2-isopopylaminoethanol sulfate) can be co-administered at a dosage range of about 0.65 mg to about 7.8 mg per day.

In still another aspect of treating COPD, the second anti-inflammatory agent Atrovent (inhaled ipratropium or 8-azioniabicyclo (3.2.1)-octane, 3-(3-hydroxy-1-oxo-2-phenylpropoxy)-8-methyl-8-(1-methylethyl)-bromide, monohydrate) can be co-administered at a dosage of about 36 mcg. In another aspect, Atrovent can be administered up to about four times per day.

vi) Allergies

Allergies, such as allergic rhinitis (hay fever), can cause inflammatory responses or reactions ranging from mild itchy skin and watery eyes to life-threatening anaphylactic shock. An allergic reaction occurs when an allergic individual exhibits an abnormally high sensitivity to a substance or compound, resulting in the stimulation of the individual's immune system to react against an allergen. Allergic reactions can be classified into four general types including, Type I, Type II, Type III, and Type IV.

Methods of reducing inflammation in a subject such as those including the co-administration of a DHEA congener and a second anti-inflammatory agent to the subject can be used in treating and/or preventing allergies, allergic reactions, and allergic rhinitis (hay fever), or the adverse symptoms thereof. Such second anti-inflammatory agents that can be delivered with a DHEA congener for the treatment of allergic rhinitis (hay fever) can include topical glucocorticoids, beclomethasone, budesonide, flunisolide, fluticasone, triamcinolone, acetonide, cromolyn, α-adrenergic agonists, pseudoephedrine, phenylephrine, phenylpropanolamine, fexofenadine hydrochloride, loratadine, and combinations thereof. Specifically, the following second anti-inflammatory agent dosage amounts can be co-administered with a DHEA congener for treating allergies.

In one aspect of the present invention, the second anti-inflammatory agent Nasarel (flunisolide or 6α-fluoro-11β,16α,17,21, tetrahydroxypregna-1,4-diene-3,20-dione cyclic 16,17-acetal hemihydrate) can be co-administered at a dosage range of about 50 mcg to about 400 mcg per day.

In another aspect of treating allergies, the second anti-inflammatory agent Allegra (fexofenadine hydrochloride or (+/−)-4-[1-hydroxy-4-[4-(hydroxydiphenyl-methyl)-1-piperidinyl]-butyl]-α,α-dimethyl benzeneacetic acid hydrochloride) can be co-administered at a dosage of about 30 mg to about 60 mg per day.

In yet another aspect of treating allergies, the second anti-inflammatory agent Flonase (fluticasone propionate or S-fluoromethyl-6α,9-difluoro-11β-17-dihydroxy-16α-methyl-3-oxoandrosta-1,4-diene-17β-carbothioate, 17-propionate) can be co-administered at a dosage range of about 50 mcg to about 200 mcg per day.

In still another aspect of treating allergies, the second anti-inflammatory agent Claritin (loratadine or ethyl-4-(8-chloro-5,6-dihydro-11H-benzo[5,6]cyclohepta[1,2-b]pyridin-11-ylidene)-1-piperidinecarboxylate) can be co-administered at a dosage range of about 5 mg to about 10 mg per day.

vii) Rheumatic Fever

Rheumatic fever is an inflammatory disease that can develop during or after an infection with streptococcus bacteria. It can additionally involve inflictions to the heart, joints, skin, and brain. Rheumatic fever can be responsible for many cases of damaged heart valves, which is commonly refered to as rheumatic heart disease.

Methods of reducing inflammation in a subject such as those including the co-administration of a DHEA congener and a second anti-inflammatory agent to the subject can be used in treating and/or preventing rheumatic heart disease. More specifically, the DHEA congener can be co-administered with the second anti-inflammatory agent to treat and/or prevent rheumatic fever and/or rheumatic heart disease. Such second anti-inflammatory agents that can be delivered with a DHEA congener for the treatment rheumatic fever and rheumatic heart disease include antibiotics, acetylsalicylic acid, corticosteroids, and, cyclosporine. In a few specific embodiments, the following second anti-inflammatory agent dosage amounts can be co-administered with a DHEA congener for treating rheumatic fever and/or rheumatic heart disease.

In one aspect of the present invention, the second anti-inflammatory agent Depo-medrol (methylprednisolone acetate or pregna-1,4-diene-3,20-dione, 21-(acetyloxy)-11,17-dihydroxy-6-methyl-, (6α,11β)) can be co-administered at a dosage range of about 40 mg to 120 mg per week.

In another aspect of treating rheumatic fever, the second anti-inflammatory agent Bicillin C-R (penicillin G or (2S,5R,6R0-3,3-dimethyl-7-oxo-6-(2-phenylacetamido)-4-thia-1-azabicyclo [3.2.0]heptane-2-carboxylic acid with either N,N′-dibenzylethylenediamine (2:1), tetrahydrate or 2-(diethylamino)ethyl p-amino-benzoate (1:1) monohydrate) can be co-administered at a dosage range of about 600,000 units to about 2,400,000 units one time.

In yet another aspect of treating rheumatic fever, the second anti-inflammatory agent Zithromax (azithromycin or (2R,3S,4R,5R,8R,10R,11R,12S,13S,14R)-13-[(2,6-dideoxy-3-C-methyl-3-O-methyl-α-L-ribo-hexopyranosyl)oxy]-2-ethyl-3,4,10-trihydroxy-3,5,6,8,10,12,14-heptamethyl-11-[[3,4,6-trideoxy-3-(dimethylamino)-β-D-xylo-hexopyranosyl]oxy]-1-oxa-6-azacyclopentadecan-15-1) can be co-administered at a dosage of about 250 mg per day.

In still another aspect of treating rheumatic fever, the second anti-inflammatory agent Gengraf (cyclosporine or [R-[R*-(E)]]-cyclic-(L-alanyl-D-alanyl-N-methyl-L-leucyl-N-methyl-L-leucyl-N-methyl-L-valyl-3-hydroxy-N,4-dimethyl-L-2-amino-6-octenoyl-L-α-amino-butyryl-N-methylglycyl-N-methyl-L-leucyl-L-valyl-N-methyl-L-leucyl) can be co-administered at a dosage range of about 7 mg/kg to about 10 mg/kg of body weight per day.

viii) Bleeding Disorders

Many bleeding disorders can be exacerbated by an improper immunological response that induces inflammation at the bleeding site. Thrombocytopenia, which is a bleeding disorder that arises from a diminished platelet count, exemplifies such bleeding disorders. As such, any inflammation around a bleeding site can cause the bleeding to increase. Thus, inhibition of inflammation can treat some of these bleeding disorders.

As such, methods of reducing inflammation in a subject such as those including the co-administration of a DHEA congener and a second anti-inflammatory agent to the subject can be used in treating and/or preventing some bleeding disorders. Such second anti-inflammatory agents that can be delivered with a DHEA congener for the treatment of bleeding disorders such as thrombocytopenia include indometacin, anti-malarials, penicillamine (D-penicillamine), sulfasalazine, cyclosporine, acetylsalicylic acid, sulfasalazine, cyclosporine, colchicines, and azathioprine. Specifically, the following second anti-inflammatory agent dosage amounts can be co-administered with a DHEA congener for treating bleeding disorders.

In one aspect of the present invention, the second anti-inflammatory agent Prelone (prednisolone or 11β,17,21-trihydrox-pregna-1,4 diene-3,20 dione) can be co-administered at a dosage range of about 5 mcg to about 60 mcg per day.

In another aspect of treating a bleeding disease, the second anti-inflammatory agent Imuran (azathioprine or 6-[(1-methyl-4-nitro-1H-imidazol-5yl)thio]-1H-purine) can be co-administered at a dosage range of about 1 mg/kg to about 3 mg/kg of body weight per day.

ix) Kidney Inflammation

Kidney inflammation is another malady that can be treated by the methods of the present invention. For example, methods of reducing inflammation in a subject such as those including the co-administration of a DHEA congener and a second anti-inflammatory agent to the subject can be used in treating and/or preventing kidney inflammation. Such second anti-inflammatory agents that can be delivered with a DHEA congener for the treatment of kidney inflammation disorders include corticosteroid, cyclophosphamide, mycophenolate mofetil, henoxymethylpenicillin, azathioprine. In a few specific embodiments, the following second anti-inflammatory agent dosage amounts can be co-administered with a DHEA congener for treating kidney inflammation.

In one aspect of the present invention, the second anti-inflammatory agent Cellcept (mycophenolate mofetil or 2-morphlinoethyl (E)-(6)-(1,3-dihydro-4-hydroxy-6-methoxy-7-methyl-3-oxo-5-isobenzofuranyl-4-methyl-4-hexonate) can be co-administered at a dosage range of about 750 mg to about 2 grams per day.

In yet another aspect of treating kidney inflammation, the second anti-inflammatory agent Altace (ramipril or (2S3αS,6αS)-{[(S)-N-[(S)-1-Carboxy-3-phenyl-propyl]]alanyl]octahydrocyclopenta [b]pryrrole-2carboxylic acid, 1-ethyl ester) can be co-administered at a dosage range of about 1.25 mg to about 5 mg per day.

In still another aspect of treating kidney inflammation, the second anti-inflammatory agent Vioxx (rofecoxib or 4-[4-(methylsulfonyl)phenyl]-3-phenyl-2(5H)-furanone) can be co-administered at a dosage of about 25 mg per day.

x) Lupus

Lupus is another malady that can be treated by methods of the present invention. For example, methods of reducing inflammation in a subject such as those including the co-administration of a DHEA congener and a second anti-inflammatory agent to a subject can be used in treating lupus, or adverse symptoms associated therewith. Such second anti-inflammatory agents that can be delivered with a DHEA congener for the treatment of lupus include azathioprine, corticosteroid, cyclophosphamide, phenoxymethylpenicillin, cevimeline hydrochloride, naproxen, corticosteroids, prednisolone, and combinations thereof. In a few specific embodiments, the following second anti-inflammatory agent dosage amounts can be co-administered with a DHEA congener for treating lupus.

In one aspect of the present invention, the second anti-inflammatory agent Evoxac (cevimeline hydrochloride or cis-2′-methylspiro [1-azabicyclo-[2.2.2] octane-3,5′-[1,3] oxathiolane] hydrochloride, hydrate (2:1)) can be co-administered at a dosage of about 90 mg per day.

In another aspect of treating lupus, the second anti-inflammatory agent Imuran (azathioprine or 6-[(1-methyl-4-nitro-1H-imidazol-5yl)thio]-1H-purine) can be co-administered at a dosage range of about 1 mg/kg to about 3 mg/kg of body weight per day.

In yet another aspect of treating lupus, Naprosyn (naproxen or (S)-6-methoxy-α-methyl-2-napthaleneacetic acid) can be administered at a dosage range of about 125 mg to about 1375 mg.

In still another aspect of treating lupus, the second anti-inflammatory agent Prelone (prednisolone or 11β,17,21-trihydrox-pregna-1,4 diene-3,20 dione) can be co-administered at a dosage of about 5 mcg to 60 mcg per day.

xi) Atopic Dermatitis

Atopic dermatitis is another malady that can be treated by methods of the present invention. For example, methods of reducing inflammation in a subject such as those including the co-administration of a DHEA congener and a second anti-inflammatory agent to the subject can be used in treating and/or preventing atopic dermatitis. Such second anti-inflammatory agents that can be delivered with a DHEA congener for the treatment of atopic dermatitis include clobetasol propionate, corticosteroids, antihistamines, antibiotics, corticosteroid skin creams, oral prednisone, tacrolimus, hydrocortisone, adrenocorticosteroid, glucocorticoid, triamcinolone, hydroxyzine, prednisolone, lodoxamide, ciprofloxacin, and ascomycin. In a few specific embodiments, the following second anti-inflammatory agent dosage amounts can be co-administered with a DHEA congener for treating atopic dermatitis. In one aspect of the present invention, the second anti-inflammatory agent Anusol-HC 2.5% (hydrocortisone cream or Pregn-4-ene-3,20-dione, 11,17,21, trihydroxy-, (11β)) can be applied from about 2 to about 4 times per day.

In another aspect of treating atopic dermatitis, the second anti-inflammatory agent Prelone (prednisolone or 11β,17,21-Trihydrox-pregna-1,4 diene-3,20 dione) can be co-administered at a dosage range of about 5 mcg to about 60 mcg per day.

In yet another aspect of treating atopic dermatitis, the second anti-inflammatory agent Cipro (ciprofloxacin hydrochloride or 1-cyclopropyl-6-fluoro-1,4-dihydro-4-oxo-7-(1-piperazinyl)-3-quinolinecarboxylic acid) can be co-administered at a dosage range of about 250 mg to about 750 mg.

In still another aspect of treating atopic dermatitis, the second anti-inflammatory agent Protopic (tacrolimus or [3S-[3R*[E(1S*,3S*,4S*)], 4S*,5R*,8S*,9E,12R*,14R*,15S*,16R*,18S*,19S*,26aR*]]-5,6,8,11,12,13,14,15,16,18,19,24,25,26,26α-hexadecahydro-5,19-dihydroxy-3-[2-(4-hydroxy-3-methoxycyclohexyl-)-1-methylethenyl]]-14,16-dimethoxy-4,10,1218-tetramethyl-8-(2-propenyl)-15,19-epoxy-3H-pyrido{2,1-c][1,4]oaazacyclotricosine-1,7,20,21(4H, 23H)-tetrone, monohydrate) can be co-administered at a dosage of about 0.03% to about 0.1%.

xii) Tissue Necrosis

Tissue necrosis is another malady that can be treated by methods of the present invention. As such, methods of reducing inflammation in a subject in accordance with embodiments of the present invention can be used to treat and/or prevent tissue necrosis. Such second anti-inflammatory agents that can be delivered with a DHEA congener for the treatment of tissue necrosis include dopamine, norepinephrine, and phenylephrine. In one embodiment, the following second anti-inflammatory agent dosage amounts can be co-administered with a DHEA congener for treating tissue necrosis.

In one aspect of the present invention, the second anti-inflammatory agent Permax (dopamine/pergolide mesylate or 8β-[(Methylthio)methyl]-6-propylergoline monomethanesulfonate) can be co-administered at a dosage of about 0.05 to about 5 mg per day.

xiii) Tuberculosis

Tuberculosis is another malady that can be treated by methods of the present invention. As such, methods of reducing inflammation in a subject in accordance with embodiments of the present invention can be effective in treating tuberculosis. Such second anti-inflammatory agents that can be delivered with a DHEA congener for the treatment of tuberculosis include isoniazid, rifampin, pyrazinamide, pyridoxine, ethambutol, streptomycin, rifabutin, rifapentine, ethionamide, cycloserine, capreomycin, amikacin, kanamycin, thiacetazone, quinolones, ofloxacin, ciprofloxacin, sparfloxacin, macrolides, clarithromycin, clofazimine, amoxycillin, clavulanic acid, monoclonal antibodies (infliximab), and combinations thereof. In a few embodiments, the following second anti-inflammatory agent dosage amounts can be co-administered with a DHEA congener for treating tuberculosis.

In one aspect of the present invention, the second anti-inflammatory agent Rifadin (rifampin or 5,6,9,17,19,21,-hexahydroxy-23-methocxy-2,4,12,16,20,22-hepamethyl-8-[N-(4-methyl-1-piperazinyl)formindioyl]2,7-(epoxypentadeca[1,11,13]trienimino)naphtha[2,1-b]furan-1,11(2H)-dione 21 acetate) can be co-administered at a dosage range of about 10 mg/kg to about 600 mg/kg of body weight per day.

In another aspect of treating tuberculosis, the second anti-inflammatory agent Amoxil (amoxicillin or (2S,5R,6R)-6-[R-(−)-2amino-2-(p-hydroxyphenyl)acetamido]-3,3-dimethyl-7-oxo-4-thia-1-azabicyclo-[3.2.0]heptane-2-carboxylic acid trihydrate) can be co-administered at a dosage range of about 10 mg/kg to about 1500 mg/kg of body weight per day.

In yet another aspect of treating tuberculosis, the second anti-inflammatory agent Cipro (ciprofloxacin or 1-cyclopropyl-6-fluoro-1,4-dihydro-4-oxo-7-(1-piperazinyl)-3-quinolinecarboxylic acid) can be co-administered at a dosage of about 400 mg to about 800 mg per day.

In still another aspect of treating tuberculosis, the second anti-inflammatory agent Mycobutin (rifabutin or (9S,12E,14S,15R,16S,17R,18R,19R,20S,21S,22E,24Z)-6,16,18,20-tetrahydroxy-1′-isobutyl-14-methoxy-7,9,15,17 19,2,25-heptamethyl-spiro[9,4-(epoxypentadeca[1,11,13] trienimino-2H-furo[2′, 3′:7,8]napth[1,2-d]imidazole-2,4′-piperidine]-5,10,26-(3H, 9H)-trione-16-acetate) can be co-administered at a dosage range of about 150 mg to about 300 mg per day.

xiv) Chronic Cholecystitis

Chronic cholecystitis is another malady that can be treated by methods of the present invention. Thus, methods of reducing inflammation in a subject in accordance with embodiments of the present invention can be effective for treating and/or preventing chronic cholecystitis. Such second anti-inflammatory agents that can be delivered with a DHEA congener for the treatment of chronic cholecystitis include gentamicin, metronidazole. In one specific embodiment, the following second anti-inflammatory agent dosage amounts can be co-administered with a DHEA congener for treating chronic cholecystitis.

In one aspect of the present invention, the second anti-inflammatory agent Metrogel (metronidazole or 2-methyl-5-nitro-1H-imidazole-1-ethanol) at a concentration of about 0.75% w/w can be applied about 3 times per day.

xv) Bronchiectasis

Bronchiectasis is another malady that can be treated by methods of the present invention. As such, methods of reducing inflammation in a subject in accordance with embodiments of the present invention can be effectively used for treating and/or preventing bronchiectasis. Such second anti-inflammatory agents that can be delivered with a DHEA congener for the treatment of bronchiectasis include ampicillin, amoxicillin, tetracycline, and combinations thereof. Specifically, the following second anti-inflammatory agent dosage amounts can be co-administered with a DHEA congener for treating bronchiectasis.

In one aspect of the present invention, the second anti-inflammatory agent Amoxil (amoxicillin or (2S,5R,6R)-6-[R-(−)-2amino-2-(p-hydroxyphenyl)acetamido]-3,3-dimethyl-7-oxo-4-thia-1-azabicyclo-[3.2.0]heptane-2-carboxylic acid trihydrate) can be co-administered at a dosage range of about 10 mg/kg to about 1500 mg/kg of body weight per day.

In another aspect of treating bronchiectasis, the second anti-inflammatory agent Achromycin V (tetracycline or (4S,4αS,5αS,12αS)-4-(Dimethylamino)-1,4,4a,5,5a,6,11,12a-octahydro-3,6,10,12,12a-pentahydroxy-6-methyl-1,11-dioxo-2-naphthacenecarboxamide) can be co-administered at a dosage range of about 1 gram to about 2 grams per day.

xvi) Hashimoto's Thyroiditis

Hashimoto's thyroiditis is another malady that can be treated by methods of the present invention. As such, methods of reducing inflammation in a subject in accordance with embodiments of the present invention can be used in treating and/or preventing Hashimoto's thyroiditis. Such second anti-inflammatory agents that can be delivered with a DHEA congener for the treatment of hashimoto's thyroiditis include fluoxetine, d-amphetamine, levothyroxine, Eltroxin, Synthroid, or combinations thereof. Specifically, the following second anti-inflammatory agent dosage amounts can be co-administered with a DHEA congener for treating hashimoto's thyroiditis.

In one aspect of the present invention, the second anti-inflammatory agent Sarafem (fluoxetine hydrochloride or (±)N-Methyl-3-phenyl-3-[(a,a,a-trifluoro-p-tolyl)oxy]propylamine hyrdrochloride) can be co-administered at a dosage range of about 20 mg to about 60 mg per day.

In another aspect of treating hashimoto's thyroiditis; the second anti-inflammatory agent Eltroxin (levothryoxine sodium or L-O-(4-Hydroxy-3,5diiodophenyl)-3,5-diodo-L-tyrosine monosodium salt hydrate) can be co-administered at a dosage range of about 50 mcg to about 200 mcg per day.

xvii) Pneumoconiosis

Pneumoconiosis is another malady that can be treated by methods of the present invention. Thus, methods of reducing inflammation in a subject in accordance with embodiments of the present invention can be used in treating and/or preventing pneumoconiosis, including silicosis. Such second anti-inflammatory agents that can be delivered with a DHEA congener for the treatment of pneumoconiosis and silicosis include bronchodilators, albuterol, epinephrine, ipratropium, metaproterenol, terbutaline, and combinations thereof. In certain embodiments, the following second anti-inflammatory agent dosage amounts can be co-administered with a DHEA congener for treating Pneumoconiosis.

In one aspect of the present invention, the second anti-inflammatory agent Combivent (ipratropium bromide or 8-azinabicyclo[3.2.1]octane, 3-(3-hydroxy-1-oxo-2-phenylpropoxy)-8-methyl-8-(1-methylethyl)-bromide, monohydrate (endo, syn)-, (+/−)-, in conjunction with albuterol sulfate or 1,3-benzene-dimethanol, α′-[[(1,1-dimethylethyl)amino] methyl]-4-hydroxy, sulfate (2:1)(salt), (+/−)) can be co-administered from about 2 to about 4 inhalations per day. In another aspect, the dosing regimen should not exceed about 12 inhalations in one day. In yet another aspect, the dosage amount of ipratropium bromide can be about 18 mcg. In still another aspect, the dosage amount of albuterol sulfate can be about 03 mcg, which can further be about 90 mcg of the albuterol base.

In another aspect of treating pneumoconiosis, the second anti-inflammatory agent Bricanyl (terbutaline or 1,3-Benzenediol, 5-[2-[(1,1-dimethylethyl)amino]-1-hydroxyethyl]-(9Cl)) can be co-administered at a dosage range of about 250 mcg to about 6 mg per day.

xviii) Pelvic Inflammatory Disease

Pelvic inflammatory disease (PID) is another malady that can be treated by methods of the present invention. As such, methods of reducing inflammation in a subject in accordance with embodiments of the present invention can be used for the treatment and/or prevention of PID. Such second anti-inflammatory agents that can be delivered with a DHEA congener for the treatment of PID include tetracycline, ofloxacin, cefotetan, doxycycline, clindamycin, gentamicin, metronidazole, ceftriaxone, probenecid, and combinations thereof. In a few specific embodiments, the following second anti-inflammatory agent dosage amounts can be co-administered with a DHEA congener for treating PID.

In one aspect of the present invention, Benemid (probenecid or 4-[(dipropylamino)sulfonyl]-benzoic acid) can be co-administered at a dosage range of about 25 mg/kg to about 2 grams/kg of body weight per day.

In another aspect of treating PID, the second anti-inflammatory agent Dalacin (clindamycin phosphate or Methyl 7-chloro-6,7,8-trideoxy-6-(1-methyl-trans-4-propyl-L-2-pyrrolidinecarboxamido)-1-thio-L-threo-a-D-galactooctopyranoside) can be co-administered at a dosage range of about 150 mg to about 450 mg. In another aspect, the dosing regimen can include administering a dose about every six hours.

xix) Chronic Sarcoidosis

Chronic sarcoidosis is another malady that can be treated by methods of the present invention. As such, methods of reducing inflammation in a subject in accordance with embodiments of the present invention can be used in treating and/or preventing chronic sarcoidosis. Such second anti-inflammatory agents that can be delivered with a DHEA congener for the treatment of chronic sarcoidosis include anti-TNF-α agents, corticosteroids, nonsteroidal anti-inflammatory drugs (NSAIDs), methotrexate, azathioprine, cyclophosphamide, chloroquinine phosphate, refecoxib, danazol, and combinations thereof. Specifically, the following second anti-inflammatory agent dosage amounts can be co-administered with a DHEA congener, where the appropriate dosing regimens can be included during the co-administration with the DHEA congener for treating chronic sarcoidosis.

In one aspect of the present invention, the second anti-inflammatory agent Vioxx (rofecoxib or 4-[4-(methylsulfonyl)phenyl]-3-phenyl-2(5H)-furanone) can be co-administered at a dosage of about 25 mg per day.

In another aspect of treating chronic sarcoidosis, the second anti-inflammatory agent methotrexate, or N-[4-[[2,4-diamino-6-pteridinyl)methyl-amino]benzoyl]-L-glutamic acid can be co-administered at a dosage range of about 7.5 mg to about 15 mg per week.

In yet another aspect of treating chronic sarcoidosis, the second anti-inflammatory agent Imuran (azathioprine or 6-[(1-methyl-4-nitro-1H-imidazol-5yl)thio]-1H-purine) can be co-administered at a dosage range of about 1 mg/kg to about 3 mg/kg of body weight per day.

xx) Pancreatitis

Pancreatitis is another malady that can be treated by methods of the present invention. Methods of reducing inflammation in a subject, such as those including the co-administration of a DHEA congener and a second anti-inflammatory agent to the subject, can be used in treating and/or preventing pancreatitis. Such second anti-inflammatory agents that can be delivered with a DHEA congener for the treatment and/or prevention of pancreatitis include COX-2 inhibitors, such as celecoxib, rofecoxib, valdecoxib, parecoxib, lumiracoxib, and combinations thereof.

xxi) Cardiovascular Disease

The current dogma suggests that the events surrounding atherosclerosis involves plaque formation followed by a localized inflammatory event. Various inflammatory markers are elevated in cardiovascular disease. Some of these are C-reactive protein (CRP), interleukin-6, and monocyte chemoattractant protein-1 (MCP-1). DHEA can be effective alone in inhibiting or reducing each of these pro-inflammatory cytokine and chemokines. In addition, 3-hydroxy-methylglutaryl coenzyme A (HMG-CoA) reductase is a key enzyme in cholesterol biosynthesis. Statins are HMG-CoA reductase inhibitors, and thus, are often used in ischemic heart disease.

The anti-inflammatory effect of statins has been demonstrated in in vivo models of atherosclerosis and in human carotid stenosis. The mechanism for the anti-inflammatory effect of statins is throught the reduction of the pro-inflammatory effect of CRP on endothelial cells, IL-6, and MCP-1. Additionally, statins have anti-inflammatory effects by reduction of cellular adhesion molecules, inhibition of NF-κB, and reduction of monocyte chemotaxis. In addition to cardiovascular disease, statins are implicated as beneficial for used to treat other maladies as well. This list includes multiple sclerosis, Alzheimer's disease, squamous cell carcinoma, transplantation, and arthritis. Thus, the co-administration of a DHEA congener and a statin can more effectively reduce inflammation in treating cardiovascular disease, as well as other diseases associated with inflammation.

Statins that can be co-administered with DHEA congeners include, but are not limited to atorvastatin, simvistatin, lovastatin, fluvastatin, and pravastatin. As new statins are discovered, one skilled in the art could modify this list accordingly. Statin dosages for co-administration with the dosages of DHEA congeners described above, e.g., preferably from about 10 mg/day to about 100 mg/day, can be in the range of about 10 mg/day to about 80 mg/day to achieve the desired anti-inflammatory effect.

Alternatively, nicotinamide, nicotinic acid, or “niacin,” the amide form of niacin (vitamin B3), is a precursor for the coenzyme beta-nicotinamide adenine dinucleotide (NAD+) and is considered to be necessary for cellular function and metabolism. Niacin has multifarious lipoprotein and anti-atherothrombosis effects that improve endothelial function, reduce inflammation, increase plaque stability, and diminish thrombosis. Further, nicotinamide is an agent that is beneficial for modulating cellular plasticity, longevity, and inflammatory microglial function. The capacity of nicotinamide to govern not only intrinsic cellular integrity, but also extrinsic cellular inflammation rests with the modulation of a host of cellular targets that involve protein kinase B, glycogen synthase kinase-3 beta (GSK-3 beta), Forkhead transcription factors, mitochondrial dysfunction, poly(ADP-ribose) polymerase, cysteine proteases, and microglial activation. Niacin includes, but is not limited to, nicobid, nicolar, niacor, and slo-niacin. Niacin can be delivered with DHEA in the ranges described above, e.g., preferably from about 10 mg/day to about 100 mg/day, in the range of about 50 mg/day to about 6000 mg/day.

C) Anti-TNF-α Agents for Administration with DHEA Congener

As described with respect to some of the previously cited diseases or conditions, DHEA congeners can be used in effective dosing regimens for reducing inflammation, especially when co-administered with an anti-TNF-α agent. Such anti-TNF-α agents can include certain monoclonal antibodies, which may be chimeric anti-TNF-α (IgG1) monoclonal antibodies. For example, in one embodiment, such an anti-TNF-α agent that can be used includes infliximab. In accordance with embodiments of the present invention, the DHEA congener and the anti-TNF-α agent can each be administered to a subject at a therapeutically effective amount to preemptively inhibit inflammation, reduce inflammation, and/or otherwise treat the subject experiencing inflammation. In one aspect, the DHEA congener and the anti-TNF-α agent can each be administered at a therapeutically effective dosage in combination, such that the inflammation can be reduced more than each administered alone. Though discussed herein as a separate class of pharmaceutically active agents that are effective at reducing inflammation by an alternative mechanism, anti-TNF-α agents are considered to be anti-inflammatory agents in accordance with embodiments of the present invention. Of course, any of the other anti-inflammatory agents described herein can additionally be co-administered at a therapeutically effective amount to the subject.

Without being bound to any particular theory, it is believed that the co-administration of the anti-TNF-α with DHEA can be effective by the inhibition of certain cell signaling pathways that are responsive to TNF-α. These cell signaling pathways can include NF-kappa-β, P38 MAP kinase, and combinations thereof. As such, when the NF-kappa-β and/or P38 MAP kinase cell signaling pathways are inhibited, there can be a decrease in TNF-α production and/or secretion. Thus, the systemic availability of TNF-α can become diminished, which may result in reduced inflammation.

Alternatively, there are indications that the up-regulation of TNF-α can increase the production and/or secretion of certain immune mediators that are responsive to TNF-α. These immune mediators can in turn stimulate an immunological response that increases inflammation. Accordingly, anti-TNF-α co-administered with DHEA can be effective in reducing inflammation by inhibiting the production and/or secretion of these immune mediators. These immune mediators that are responsive to TNF-α can include, without limitation, IL-1, IL-6, IL-3, G-CSF, GM-CSF, IL-10, IL-1 Ra, IL-2, IL-4, IL-8, IL-12, IL-18, IFN-γ, and combinations thereof.

In accordance with methods of the present invention, the reduction of inflammation in a subject can be utilized to treat certain diseases. These diseases may be related to the production and/or secretion of TNF-α, where the reduction of systemic TNF-α can reduce inflammation in the subject. Examples of diseases, some of which are repetitive to those described previously, that can be treated by the methods described herein include rheumatoid arthritis, psoriatic arthritis, psoriasis, sarcoidosis, Adult Still's disease, severe acute ulcerative colitis, spondyloarthropathies, ankylosing spondylitis, Bechet's syndrome, Crohn's disease, orofacial Crohn's disease, uveitis, HIV-1-associated psoriatic arthritis, gravt-vs-host disease, advanced heart failure, common variable immunodeficiency, Wegener's granulomatosis, sepsis, pyoderma gangrenosum, subcorneal pustular dermatosis, Hidradenitis suppurativa, panniculitis, and Langerhans' cell histiocytsis.

D) Preparation and Dosage Forms

Pharmaceutical compositions containing any compound of the present invention as the active ingredient can be prepared according to conventional pharmaceutical compounding techniques known to one of ordinary skill in the art. Typically, a therapeutically effective amount of the active ingredient can be admixed with a pharmaceutically acceptable carrier. The carrier may take a wide variety of forms depending on the desired route of administration, e.g., oral, intravenous, intrathecal epidural, transdermal, transbuccal, ocular, nasal, suppository. The compositions may further contain antioxidizing agents, stabilizing agents, preservatives, or the like.

Liquid carriers can be used in preparing solutions, suspensions, emulsions, syrups, elixirs and pressurized compositions. The active ingredients can be dissolved or suspended in a pharmaceutically acceptable liquid carrier such as water, cyclodextrins, an organic solvent, pharmaceutically acceptable oils, and/or fats. The liquid carrier can contain other suitable pharmaceutical additives such as solubilizers, emulsifiers, buffers, preservatives, sweeteners, flavoring agents, suspending agents, thickening agents, coloring agents, viscosity regulators, stabilizers and/or osmo-regulators. Suitable examples of liquid carriers for oral administration can include water, alcohols (including monohydric alcohols and polyhydric alcohols, e.g., glycols) and their derivatives, oils (e.g., peanut oil, sesame oil, olive oil, and coconut oil), and combinations of the above. Compositions comprising such carriers and adjuvants may be formulated using well-known conventional materials and methods.

A solid carrier can be formulated into capsules, pills, tablets, lozenges, melts, or powders. A solid carrier can include starches, sugars, bicarbonates, diluents, granulating agents, disintegrating, and/or dispersing agents. The formulations can include one or more substance(s) which may also act as flavoring agents, lubricants, solubilizers, suspending agents, fillers, glidants, compression aids, binders, or tablet-disintegrating agents, for example. In powders, the carrier can be a finely divided solid which is in an admixture with the finely divided active ingredients. The carrier and drug can form a single composite with drug adsorbed to its surface that effectively enhances the rate of dissolution in the gastrointestinal tract. The powders and/or tablet can contain up to 100 wt % of the active ingredients, though typically this will not be the case, and can be formulated for immediate and/or sustained release of the active ingredient.

With respect to tablets, the active ingredients can be mixed with a carrier having the necessary compression properties in suitable proportions and compacted in the shape and size desired. Exemplary forms can include dry powder compaction tablets, micro-particulate systems, e.g., wherein the active ingredient is spray-dried onto a scaffold particle, and hard or soft-gel capsules. In one embodiment, the tablets can be optionally covered with an enteric coating, which remains intact in the stomach, but will dissolve and release the contents of the tablet once it reaches the small intestine. Alternatively, the tablets can be formulated to enhance gastric uptake to avoid first pass effect in the liver following intestinal absorption.

The composition can include one or more sustained or controlled release excipient(s) such that a slow, sustained, or constant release of the active ingredients can be achieved. A wide variety of suitable excipients are known in the art. Such sustained/controlled release excipients and systems are described, for example, in U.S. Pat. Nos. 5,612,053; 5,554,387; 5,512,297; 5,478,574; and 5,472,711, each of which is incorporated by reference herein. If desired, the pharmaceutical composition can be formulated to provide a pulse dose of the active ingredient. A variety of pulse-dose systems, which provide low or high-pulsed doses, are known in the art. In another embodiment of the invention, the pharmaceutical can be formulated to provide direct and/or targeted delivery of the active ingredient to a specific anatomic site or sites within the gastrointestinal tract; e.g., the duodenum, jejunum, ileum, cecum and/or colon. Methods for providing targeted delivery of pharmaceuticals to specific tissues or organs within a mammalian host are well known in the art.

To achieve a therapeutic level in systemic circulation, a compound, i.e. active agent(s), can be formulated using standard techniques to form a composition having a high bioavailability of the active agents in order to meet the desired therapeutic blood levels. The active agent(s), a complex of the active agent(s) and cyclodextrin, or the active agent(s) in a nanoparticle delivery system may be dissolved in a pharmaceutical carrier and administered as either a solution or a suspension. Cyclodextrins of all classes (alpha, beta and gamma) and their substituted or derivatized forms can be used, as well as mixtures thereof. In one aspect of the present invention, a complex of the active agent(s) with a cyclodextrin or the active agent in a nanoparticle delivery system can be used. In another aspect, a complex of the active agent(s) and a cyclodextrin, such as a 2-hydroxypropyl β-cyclodextrin, can be prepared in accordance with U.S. Pat. No. 4,727,064 and/or European Patent No. 0 149 197, each incorporated herein by reference. The use of the compound as part of a cyclodextrin complex or nanoparticle delivery system can allow for the preparation of both parenteral and oral solutions and oral solid dosage forms with high concentration of active agent. Illustrative of suitable carriers include water, saline, dextrose solutions, fructose solutions, ethanol, or oils of animal, vegetative or synthetic origin. The carrier may also contain other ingredients including, for example, preservatives, suspending agents, solubilizing agents, buffers, and/or the like. When the compounds are being administered intrathecally, they or their cyclodextrin complexes or nanoparticle delivery systems may also be dissolved in cerebrospinal fluid.

The active agent can be administered in a therapeutically effective amount. The actual amount administered, and the rate and time-course of administration, can depend on the nature and severity of the condition being treated. Thus, it may be desirable to administer the highly bioavailable complex of the active agent at several intervals during a dosing regimen to maintain blood levels at the therapeutic index. For example, depending on the dosing regimen, it can be preferable to administer the formulated active agent two to four times per day, and more preferably to administer it two to three times per day. Alternatively, one might use an oral controlled release method to meter the drug available for absorption over a 24 hour period, thereby necessitating only a single dose per day. Prescription of treatment, e.g. decisions on dosage, timing, periods of administration, drug selection, etc., can be within the responsibility of general practitioners or specialists, and typically can take into account the disorder to be treated, the condition of the individual patient, the site of delivery, the method of administration, and other factors known to practitioners.

As will be appreciated by those of skill in the art, the form of the pharmaceutical composition of the active agent(s) and the mode of administration will determine the dose of the active agent to be delivered. A factor to consider in determining the proper dose to meet the desired peak blood levels is the bioavailability of the active agent in the pharmaceutical composition, i.e., the availability of the active agent for raising blood levels of DHEA or DHEA equivalent. For example, the bioavailability of the active agent(s) in a pharmaceutical composition delivered intravenously can be greater than that for the same pharmaceutical composition delivered orally. Thus, a lower dose of a pharmaceutical composition containing the active agent(s) can be administered intravenously than that which would be used orally. For oral administration of active agent(s) with low water solubility, it is well recognized that co-formulation of the agent(s) with a substance that accelerates dissolution or enhances solubility can increase the active agent's bioavailability. For example, active agent used to increase blood levels of DHEA congener or equivalent can be many times more soluble in water if complexed with a cyclodextrin than without. Similarly, the same active agent can dissolve in water much faster and have higher bioavailability if adsorbed to a high surface area particle with a large surface area. For example, suitable blood levels of the DHEA congener can be achieved by the administration of 100 mg of a cyclodextrin-DHEA complex intravenously, 600 mg of a cyclodextrin-DHEA complex orally, or 500 mg of DHEA in a nanoparticle delivery system orally. As will also be appreciated by those of skill in the art, the form of the pharmaceutical composition of the DHEA congener and the second active agent can depend on the intended mode of administration, which in turn will depend on the location and nature of the disorder to be treated. Accordingly, delivery to the gastrointestinal tract, e.g., for treatment of gastrointestinal mucositis, peptic ulcers and inflammatory bowel diseases, such as Crohn's disease, ulcerative colitis, indeterminate colitis, and infectious colitis, can be in the form of oral solutions, gels, suspensions, tablets, capsules, and the like.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Reducing Inflammation of Rheumatoid Arthritis

A human subject having rheumatoid arthritis is treated by the following procedure. In this example, a formulation having 100 mg of DHEA (dehydroepiandrosterone) and 25 mg of rofecoxib is provided. The formulation is administered orally to a human subject experiencing inflammation of the finger joints caused by rheumatoid arthritis. The dosing regimen includes one dose of the formulation in the morning of each day. After treatment, the inflammation of the finger joints can be diminished.

### Example 2

### Treating the Symptoms of Lupus

A human subject previously diagnosed with lupus is treated by the following procedure. In this example, a formulation having 75 mg of DHEA (dehydroepiandrosterone) and 30 mg of prednisolone is provided. The formulation including is administered orally to a human subject diagnosed with lupus that is also currently experiencing swollen and painful joints. The dosing regimen includes administering the formulation one time per day in the morning. After treatment, the pain associated with lupus is diminished.

### Example 3

### Effect of DHEA and/or Sulfasalazine on TNF-α In Vivo

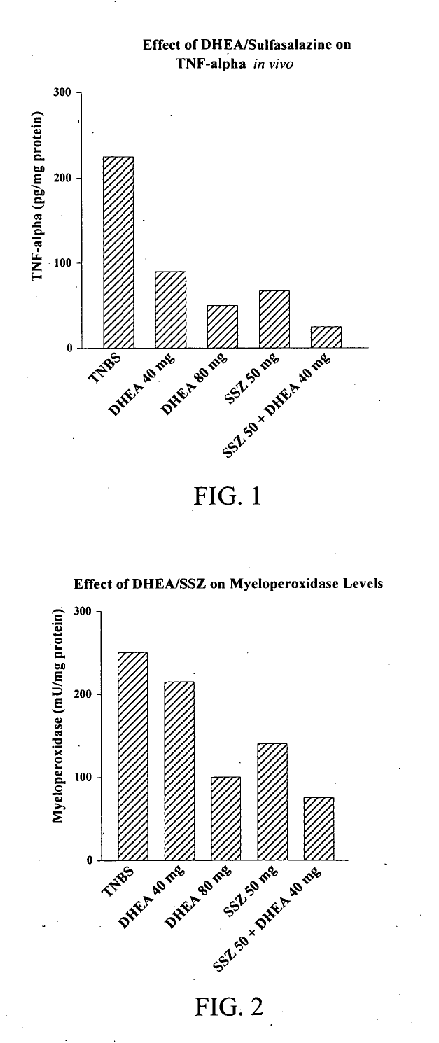

The effects of DHEA and/or sulfasalazine on a TNBS IBD animal model was studied by administering DHEA alone (40 mg/kg/day or 80 mg/kg/day), sulfasalazine (SSZ) alone (50 mg/kg/day), and a combination of DHEA and sulfasalazine (40 mg/kg/day DHEA with 50 mg/kg/day sulfasalazine). The intrarectal administration of TNBS (2,4,6-trinitrobenzene sulfonic acid) in mice/rats is a well-characterized animal model for human inflammatory bowel disease (IBD). The TNF-α levels (pg/mg protein, 3 days after challenge with intracolonic TNBS) for the treated animals was collected and compared to an untreated TNBS animal model. FIG. 1 illustrates the results. As can be seen from FIG. 1, a combination of DHEA with sulfasalazine works in a gold standard IBD animal model for reducing TNF-α levels better than either DHEA or sulfasalazine alone.

### Example 4

### Effect of DHEA and/or Sulfasalazine Myeloperoxidase Levels

The effects of DHEA and/or sulfasalazine on a TNBS IBD animal model was studied by administering DHEA alone (40 mg/kg/day or 80 mg/kg/day), sulfasalazine (SSZ) alone (50 mg/kg/day), and a combination of DHEA and sulfasalazine (40 mg/kg/day DHEA with 50 mg/kg/day sulfasalazine). The intrarectal administration of TNBS (2,4,6-trinitrobenzene sulfonic acid) in mice/rats is a well-characterized animal model for human inflammatory bowel disease (IBD). The myeloperoxidase levels (mU/mg protein, 3 days after challenge with intracolonic TNBS) for the treated animals was collected and compared to an untreated TNBS animal model. Myeloperoxidase levels are a measure of white cell infiltrate to the site of inflammation. FIG. 2 illustrates the results. As can be seen from FIG. 2, a combination of DHEA with sulfasalazine works in a gold standard IBD animal model for reducing myeloperoxidase levels better than either DHEA or sulfasalazine alone.

### Example 5

### Effect of DHEA and/or Ibuprofen on Arthritis

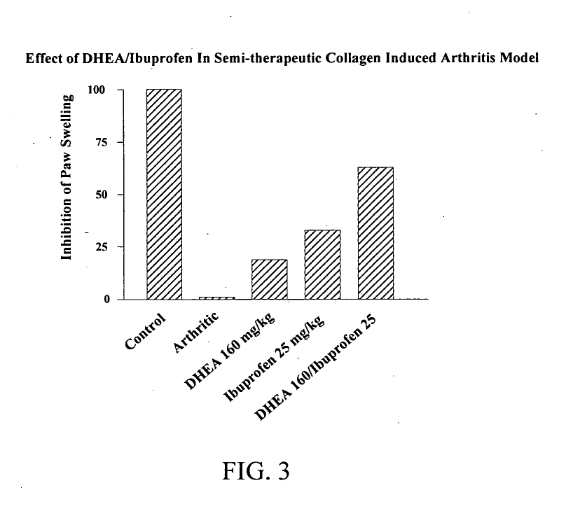

The effects of DHEA and/or ibuprofen, each alone as well as in combination was studied using in vivo paw swelling tests in a collagen induced arthritis animal models, the results of which are shown in FIG. 3. A control animal and an arthritic animal were compared and characterized in FIG. 3. Additionally, a first animal model was administered DHEA alone (160 mg/kg), another model was administered ibuprofen alone (25 mg/kg), and still another animal model was administered a combination of DHEA and ibuprofen (160 mg/kg DHEA with 25 mg/kg ibuprofen). As can be seen from FIG. 3, a combination of DHEA with ibuprofen has a greater effect than either anti-inflammatory composition used alone. In this example, in one sense, a synergistic effect is shown to have occurred, where the reduction of inflammation is greater than the sum of each anti-inflammatory composition used alone.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method of reducing inflammation in a subject, comprising co-administering therapeutically effective amounts of a DHEA congener and a second anti-inflammatory agent to the subject.

2. A method as in claim 1, wherein reducing inflammation includes preemptively co-administering the DHEA congener and the second anti-inflammatory agent to the subject to inhibit inflammation.

3. A method as in claim 1, wherein reducing inflammation includes treating the subject experiencing inflammation.

4. A method as in claim 1, wherein the co-administering step results in enhanced anti-inflammatory response compared to the administration of either the DHEA congener or the second anti-inflammatory agent alone at their respective dosages.

5. A method as in claim 1, wherein reducing inflammation includes treating rheumatoid arthritis.

6. A method as in claim 5, wherein the second anti-inflammatory agent is selected from the group consisting of leflonomide, methotraxate, rofecoxib, celecoxib, infliximab, and combinations thereof.

7. A method as in claim 5, wherein the second anti-inflammatory agent is a non-steroidal anti-inflammatory agent.

8. A method as in claim 7, wherein the non-steroidal anti-inflammatory agent is selected from the group consisting of ibuprofen, naproxen, aspirin, and combinations thereof.

9. A method as in claim 1, wherein reducing inflammation includes treating asthma.

10. A method as in claim 9, wherein the second anti-inflammatory agent is selected from the group consisting of prednisolone, ipratropium bromide, albuterol sulphate, and combinations thereof.

11. A method as in claim 1, wherein reducing inflammation includes treating an inflammatory bowel disease.

12. A method as in claim 11, wherein the inflammatory bowel disease is Crohn's disease.

13. A method as in claim 11, wherein the inflammatory bowel disease is ulcerative colitis.

14. A method as in claim 11, wherein the second anti-inflammatory agent is selected from the group consisting of budesonide, prednisolone, infliximab sulfasalazine, olsalazine sodium, daclizumab, and combinations thereof.

15. A method as in claim 1, wherein reducing inflammation includes treating multiple sclerosis.

16. A method as in claim 15, wherein the second anti-inflammatory agent is selected from the group consisting of interferon β-1a, mitoxantrone hydrochloride, glatriramer acetate, tolterodine tartrate, and combinations thereof.

17. A method as in claim 1, wherein reducing inflammation includes treating chronic obstructive pulmonary disease (COPD).

18. A method as in claim 17, wherein the second anti-inflammatory agent is selected from the group consisting of budesonide, beclomethasone dipropionate, metaproterenol sulphate, ipratropium, and combinations thereof.

19. A method as in claim 1, wherein reducing inflammation includes treating allergic rhinitis.

20. A method as in claim 19, wherein the second anti-inflammatory agent is selected from the group consisting of flunisolide, flexofenadine hydrochloride, fluticasone propionate, loratadine, and combinations thereof.

21. A method as in claim 1, wherein reducing inflammation includes treating rheumatic fever.

22. A method as in claim 21, wherein the second anti-inflammatory agent is selected from the group consisting of methylprednisolone acetate, penicillin G, azithromycin, cyclosporine, and combinations thereof.

23. A method as in claim 1, wherein reducing inflammation includes treating a bleeding disorder.

24. A method as in claim 23, wherein the bleeding disorder is thrombocytopenia.

25. A method as in claim 23, wherein the second anti-inflammatory agent is selected from the group consisting of prednisolone, danazol, and combinations thereof.

26. A method as in claim 1, wherein reducing inflammation includes treating kidney inflammation.

27. A method as in claim 26, wherein the second anti-inflammatory agent is selected from the group consisting of mycophenolate mofetil, ramipril, rofecoxib, and combinations thereof.

28. A method as in claim 1, wherein reducing inflammation includes treating lupus.

29. A method as in claim 28, wherein the second anti-inflammatory agent is selected from the group consisting of cevimeline hydrochloride, danazol, azathioprine, naproxen, prednisolone, and combinations thereof.

30. A method as in claim 1, wherein reducing inflammation includes treating atopic dermatitis.

31. A method as in claim 30, wherein the second anti-inflammatory agent is selected from the group consisting of hydrocortisone, prednisolone, ciprofloxacin hydrochloride, tacrolimus, and combinations thereof.

32. A method as in claim 1, wherein reducing inflammation includes treating tissue necrosis.

33. A method as in claim 32, wherein the second anti-inflammatory agent is selected from the group consisting of dopamine, pergolide mesylate, and combinations thereof.

34. A method as in claim 1, wherein reducing inflammation includes treating tuberculosis.

35. A method as in claim 34, wherein the second anti-inflammatory agent is selected from the group consisting of rifampin, amoxicillin, ciprofloxacin, rifabutin, and combinations thereof.

36. A method as in claim 1, wherein reducing inflammation includes treating chronic cholecystitis.

37. A method as in claim 36, wherein the second anti-inflammatory agent is selected from the group consisting of gentamicin, metronidazole, and combinations thereof.

38. A method as in claim 1, wherein reducing inflammation includes treating bronchiectasis.

39. A method as in claim 38, wherein the secondary anti-inflammatory agent is selected from the group consisting of ampicillin, amoxicillin, tetracycline, and combinations thereof.

40. A method as in claim 1, wherein reducing inflammation includes treating Hashimoto's thyroiditis.

41. A method as in claim 40, wherein the second anti-inflammatory agent is selected from the group consisting of fluoxetine hydrochloride, levothryoxine sodium, and combinations thereof.

42. A method as in claim 1, wherein reducing inflammation includes treating a pneumoconiosis.

43. A method as in claim 42, wherein the pneumoconiosis is silicosis.

44. A method as in claim 42, wherein the second anti-inflammatory agent is selected from the group consisting of ipratropium bromide, albuterol sulphate, terbutaline, and combinations thereof.

45. A method as in claim 1, wherein reducing inflammation includes treating pelvic inflammatory disease.

46. A method as in claim 45, wherein the second anti-inflammatory agent is selected from the group consisting of probenecid, clindamycin phosphate, and combinations thereof.

47. A method as in claim 1, wherein reducing inflammation includes treating chronic sarcoidoisis.

48. A method as in claim 47, wherein the second anti-inflammatory agent is selected from the group consisting of rofecoxib, methotrexate, danazol, azathioprine, and combinations thereof.

49. A method as in claim 1, wherein reducing inflammation includes treating pancreatitis.

50. A method as in claim 49, wherein the second anti-inflammatory agent is a COX-2 inhibitor.

51. A method as in claim 50, wherein the COX-2 inhibitor is selected from the group consisting of celecoxib, rofecoxib, valdecoxib, parecoxib, lumiracoxib, and combinations thereof.

52. A method as in claim 51, wherein the COX-2 inhibitor is celecoxib.

53. A method as in claim 51, wherein the COX-2 inhibitor is rofecoxib.

54. A method as in claim 1, wherein reducing inflammation includes treating cardiovascular disease.

55. A method as in claim 54, wherein the second anti-inflammatory agent is a statin.

56. A method as in claim 55, wherein the statin is selected from the group consisting of atorvastatin, simvistatin, lovastatin, fluvastatin, pravastatin, and combinations thereof.

57. A method as in claim 54, wherein the second anti-inflammatory agent is a form of nicotinamide.

58. A method as in claim 57, wherein the nicotinamide is selected from the group consisting of nicobid, nicolar, niacor, slo-niacin, and combinations thereof.

59. A method as in claim 1, wherein the DHEA congener and the second anti-inflammatory agent are each administered at a therapeutically effective dosage, such that the inflammation is reduced more than by the summation of inflammation reduction for each administered alone.

60. A method as in claim 1, wherein the subject is human.

61. A method of reducing inflammation in a subject, comprising co-administering a therapeutically effective amount of a DHEA congener and an anti-TNF-α agent to the subject.

62. A method as in claim 61, wherein the anti-TNF-α agent is a monoclonal antibody.

63. A method as in claim 62, wherein the monoclonal antibody is infliximab.

64. A method as in claim 61, wherein reducing inflammation includes preemptively co-administering the DHEA congener and the anti-TNF-α agent to the subject to inhibit inflammation.

65. A method as in claim 61, wherein reducing inflammation includes treating the subject experiencing inflammation.

66. A method as in claim 61, wherein the co-administering step results in enhanced anti-inflammatory response compared to the administration of either the DHEA congener or anti-TNF-α agent alone at their respective dosages.

67. A method as in claim 61, wherein reducing inflammation includes treating a disease selected from the group consisting of rheumatoid arthritis, psoriatic arthritis, psoriasis, sarcoidosis, Adult Still's disease, severe acute ulcerative colitis, spondyloarthropathies, ankylosing spondylitis, Bechet's syndrome, Crohn's disease, orofacial Crohn's disease, uveitis, HIV-1-associated psoriatic arthritis, gravt-vs-host disease, advanced heart failure, common variable immunodeficiency, Wegener's granulomatosis, sepsis, pyoderma gangrenosum, subcorneal pustular dermatosis, Hidradenitis suppurativa, panniculitis, Langerhans' cell histiocytsis, and cardiovascular disease.

68. A method as in claim 61, wherein the anti-TNF-α agent inhibits a cell signaling pathway that is responsive to TNF-α.

69. A method as in claim 68, wherein the cell signaling pathway is selected from the group consisting of NF-Kappa-β, P38 MAP kinase, and combinations thereof.

70. A method as in claim 61, wherein the anti-TNF-α agent inhibits an immune mediator responsive to TNF-α.

71. A method as in claim 70, wherein the immune mediator is selected from the group consisting of IL-1, IL-6, IL-3, G-CSF, GM-CSF, IL-10, IL-1 Ra, IL-2, IL-4, IL-8, IL-12, IL-18, IFN-γ, and combinations thereof.

72. A method as in claim 61, further comprising co-administering a therapeutically effective amount of a second non-TNF-α anti-inflammatory agent to the subject.

73. A method as in claim 61, wherein the subject is human.

74. A composition for reducing inflammation in a subject, comprising:
a DHEA congener;
a second anti-inflammatory agent; and
a carrier,
said composition effective for enhancing an anti-inflammatory response in the subject compared to the administration of either the DHEA congener or the second anti-inflammatory agent alone at their respective dosages.

75. A composition as in claim 74, wherein the anti-inflammatory response is a inflammation preventative response resulting from preemptively co-administering the DHEA congener and the second anti-inflammatory agent to the subject to inhibit inflammation.

76. A composition as in claim 74, wherein the anti-inflammatory response is an inflammation reducing response resulting from co-administering the DHEA congener and the second anti-inflammatory agent the subject experiencing inflammation.

77. A composition as in claim 74, wherein the second anti-inflammatory agent is a non-steroidal anti-inflammatory agent.

78. A composition as in claim 74, wherein the second anti-inflammatory agent is a COX-2 inhibitor.

79. A composition as in claim 74, wherein the second anti-inflammatory agent is selected from the group consisting of leflonomide, methotraxate, rofecoxib, celecoxib, infliximab, ibuprofen, naproxen, aspirin, prednisolone, ipratropium bromide, albuterol sulfate, budesonide, sulfasalazine, olsalazine sodium, daclizumab, interferon β-1a, mitoxantrone hydrochloride, glatriramer acetate, tolterodine tartrate, beclomethasone dipropionate, metaproterenol sulfate, ipratropium, flunisolide, flexofenadine hydrochloride, fluticasone propionate, loratadine, methylprednisolone acetate, penicillin G, azithromycin, cyclosporine, danazol, mycophenolate mofetil, ramipril, cevimeline hydrochloride, azathioprine, hydrocortisone, ciprofloxacin hydrochloride, tacrolimus, dopamine, pergolide mesylate, rifampin, ampicillin, amoxicillin, ciprofloxacin, rifabutin, gentamicin, metronidazole, tetracycline, fluoxetine hydrochloride, levothryoxine sodium, terbutaline, probenecid, clindamycin phosphate, methotrexate, statins, nicotinamides, and combinations thereof.

80. A composition for reducing inflammation in a subject, comprising:
a DHEA congener;
an anti-TNF-α agent; and
a carrier,
said composition effective for enhancing an anti-inflammatory response in the subject compared to the administration of either the DHEA congener or the anti-TNF-α agent alone at their respective dosages.

81. A composition as in claim 80, wherein the anti-TNF-α agent is a monoclonal antibody.

82. A composition as in claim 81, wherein the monoclonal antibody is infliximab.

83. A composition as in claim 80, wherein the anti-TNF-α agent inhibits a cell signaling pathway that is responsive to TNF-α.

84. A composition as in claim 83, wherein the cell signaling pathway is selected from the group consisting of NF-Kappa-β, P38 MAP kinase, and combinations thereof.

85. A composition as in claim 80, wherein the anti-TNF-α agent inhibits an immune mediator responsive to TNF-α.

86. A composition as in claim 85, wherein the immune mediator is selected from the group consisting of IL-1, IL-6, IL-3, G-CSF, GM-CSF, IL-10, IL-1Ra, IL-2, IL-4, IL-8, IL-12, IL-18, IFN-γ, and combinations thereof.
